SEQUENCE LISTING (66 sequences)

SEQ ID NO: 1 - DNA, 93 bp, synthetic construct
  source 1..93: mol_type = other DNA
gcaacgacatttgctgaacgtgtcccgcctgtcgttggcccgccgctttaacacgaagtgggcaacgatacttatcgacgatgttaggcttgg

SEQ ID NO: 2 - DNA, 91 bp, synthetic construct
  source 1..91: mol_type = other DNA
ctccaatcctccaaccatcaactcaaccaaagatcttcgcccttgccttaatatctgccatgttcgacaccaactccactcgtcttccaat

SEQ ID NO: 3 - DNA, 138 bp, synthetic construct
  source 1..138: mol_type = other DNA
agacgttgaaactggataggagagggacttgtccttcaagttgaaggwtttttcggcgctctacgttttcatataccccaaagaatgtaacagaatgtatcttawggctttatgcctataaactatatacaactttca

SEQ ID NO: 4 - DNA, 469 bp, synthetic construct
  source 1..469: mol_type = other DNA
tcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacctcaagggtgccaaagcgttggggagcaaacaggattagataccctggtagtcc

SEQ ID NO: 5 - DNA, 59 bp, synthetic construct
  source 1..59: mol_type = other DNA
cagcgtggtggcaaaaacggataccggcaaacataatgcaatcaaggtggtcacgatgc

SEQ ID NO: 6 - DNA, 152 bp, synthetic construct
  source 1..152: mol_type = other DNA
tggtttcgattcggaagcgggttatcgccgtcaggaggcattacgaaaagaaaataacattggaacaaaaatggggaacttctcattcttcagcgaagagatgactgacccgctggtcgcgttcgccgggcagtggcgaccagatctcatcg

SEQ ID NO: 7 - DNA, 149 bp, synthetic construct
  source 1..149: mol_type = other DNA
agatcattgtttttggaagctggattgaagtcagagattactctctgatgaattgaaggctttctcaatgatggtctacgttttcatatactccaatgaatgtaacagaatgtatctatatggccttgtgcctataaaacaatatacaa

SEQ ID NO: 8 - DNA, 125 bp, synthetic construct
  source 1..125: mol_type = other DNA
agtgggggagcgaggaagaaaggataaattttgtgaaaaagggggtagctgcctttcacgacgccaggggcaacaacgacaatggggaaattacgtctaccttactgaaggaatcatccactgcg

SEQ ID NO: 9 - DNA, 157 bp, synthetic construct
  source 1..157: mol_type = other DNA
gcactggcgtggtgttgtcttcactactttggacttgggttgctcctacgtgcacaaagtgagagtaaatttctcgtcaggcattatctcaagaactatcattacgattacgctcagcaagatgggggtcaagtcgccgttatcgaagcattttcca

SEQ ID NO: 10 - DNA, 151 bp, synthetic construct
  source 1..151: mol_type = other DNA
acccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgca

SEQ ID NO: 11 - DNA, 411 bp, synthetic construct
  source 1..411: mol_type = other DNA
gcatcgatgaagaacgcagcgaaatgcgataactaatgtgaattgcagaattcagtgaatcatcgagtctttgaacgcacattgcgccccctggtattccggggggcatgcctgtccgagcgtcattgctgccctcaagcccggcttgtgtgttgggtcgccgtccccctctccggggggacgggcccgaaaggcagcggcggcaccgcgtccgatcctcgagcgtatggggctttgtcacatgctctgtaggattggccggcgcctgccgacgttttccaaccattctttccaggttgacctcggatcaggtagggatacccgctgaacttaagcatatcaataagcggaggaaaagaaaccaaccgggattgcctcagtaacggcgagtgaagcggcaagagctcaaat

SEQ ID NO: 12 - DNA, 21 bp, synthetic construct
  source 1..21: mol_type = other DNA
ctccaatcctccaaccatcaa

SEQ ID NO: 13 - DNA, 21 bp, synthetic construct
  source 1..21: mol_type = other DNA
attggaagacgagtggagttg

SEQ ID NO: 14 - DNA, 20 bp, synthetic construct
  source 1..20: mol_type = other DNA
gcaacgacatttgctgaacg

SEQ ID NO: 15 - DNA, 22 bp, synthetic construct
  source 1..22: mol_type = other DNA
ccaagcctaacatcgtcgataa

SEQ ID NO: 16 - DNA, 24 bp, synthetic construct
  source 1..24: mol_type = other DNA
tcgcccttgccttaatatctgcca

SEQ ID NO: 17 - DNA, 24 bp, synthetic construct
  source 1..24: mol_type = other DNA
cgctttaacacgaagtgggcaacg

SEQ ID NO: 18 - DNA, 26 bp, synthetic construct
  source 1..26: mol_type = other DNA
gtagacgttgaaactggataggagag

SEQ ID NO: 19 - DNA, 33 bp, synthetic construct
  source 1..33: mol_type = other DNA
aaattgtatatagtttataggcataaagccwta

SEQ ID NO: 20 - DNA, 32 bp, synthetic construct
  source 1..32: mol_type = other DNA
cttgtccttcaagttgaaggatttttcggcgc

SEQ ID NO: 21 - DNA, 39 bp, synthetic construct
  source 1..39: mol_type = other DNA
tacacgacgttgtaaaacgatcctacgggaggcagcagt

SEQ ID NO: 22 - DNA, 47 bp, synthetic construct
  source 1..47: mol_type = other DNA
aggataacaatttcacacaggggactaccagggtatctaatcctgtt

SEQ ID NO: 23 - DNA, 23 bp, synthetic construct
  source 1..23: mol_type = other DNA
cgtattaccgcggctgctggcac

SEQ ID NO: 24 - DNA, 39 bp, synthetic construct
  source 1..39: mol_type = other DNA
tacacgacgttgtaaaacgacgcatcgtgaccaccttga

SEQ ID NO: 25 - DNA, 37 bp, synthetic construct
  source 1..37: mol_type = other DNA
aggataacaatttcacacaggcagcgtggtggcaaaa

SEQ ID NO: 26 - DNA, 20 bp, synthetic construct
  source 1..20: mol_type = other DNA
ccggcaaacataatgcaatc

SEQ ID NO: 27 - DNA, 39 bp, synthetic construct
  source 1..39: mol_type = other DNA
tacacgacgttgtaaaacgactggtttcgattcggaagc

SEQ ID NO: 28 - DNA, 38 bp, synthetic construct
  source 1..38: mol_type = other DNA
aggataacaatttcacacaggcgatgagatctggtcgc

SEQ ID NO: 29 - DNA, 24 bp, synthetic construct
  source 1..24: mol_type = other DNA
ccgtcaggaggcattacgaaaaga

SEQ ID NO: 30 - DNA, 30 bp, synthetic construct
  source 1..30: mol_type = other DNA
tcattgtttttggaagctggattgaagtca

SEQ ID NO: 31 - DNA, 34 bp, synthetic construct
  source 1..34: mol_type = other DNA
aaagttgtatattgttttataggcacaaggccat

SEQ ID NO: 32 - DNA, 36 bp, synthetic construct
  source 1..36: mol_type = other DNA
tactctctgatgaattgaaggctttctcaatgatgg

SEQ ID NO: 33 - DNA, 22 bp, synthetic construct
  source 1..22: mol_type = other DNA
ggagcgaggaagaaaggataaa

SEQ ID NO: 34 - DNA, 22 bp, synthetic construct
  source 1..22: mol_type = other DNA
gcagtggatgattccttcagta

SEQ ID NO: 35 - DNA, 22 bp, synthetic construct
  source 1..22: mol_type = other DNA
cgtggtgttgtcttcactactt

SEQ ID NO: 36 - DNA, 20 bp, synthetic construct
  source 1..20: mol_type = other DNA
cccatcttgctgagcgtaat

SEQ ID NO: 37 - DNA, 19 bp, synthetic construct
  source 1..19: mol_type = other DNA
acccgtcaatgggacaatc

SEQ ID NO: 38 - DNA, 22 bp, synthetic construct
  source 1..22: mol_type = other DNA
gaggtgggtcgtaatcgttatc

SEQ ID NO: 39 - DNA, 21 bp, synthetic construct
  source 1..21: mol_type = other DNA
tagctgcctttcacgacgcca

SEQ ID NO: 40 - DNA, 23 bp, synthetic construct
  source 1..23: mol_type = other DNA
tggacttgggttgctcctacgtg

SEQ ID NO: 41 - DNA, 22 bp, synthetic construct
  source 1..22: mol_type = other DNA
cgagcacgattggcgaccctat

SEQ ID NO: 42 - DNA, 41 bp, synthetic construct
  source 1..41: mol_type = other DNA
tacacgacgttgtaaaacgacgcatcgatgaagaacgcagc

SEQ ID NO: 43 - DNA, 42 bp, synthetic construct
  source 1..42: mol_type = other DNA
aggataacaatttcacacaggatttgagctcttgccgcttca

SEQ ID NO: 44 - DNA, 28 bp, synthetic construct
  source 1..28: mol_type = other DNA
cgctgaacttaagcatatcaataagcgg

SEQ ID NO: 45 - DNA, 731 bp, Psilocybe sp.
  source 1..731: mol_type = genomic DNA
ggaagtaaaagtcgtaacaaggtttccgtaggtgaacctgcggaaggatcattattgaataactttggcgtggttgtagctggccctctcgggggcatgtgctcgcccgtcatctttatatttccacctgtgcactttttgtagatcattgtttttggaagctggattgaagtcagagattactctctgatgaattgaaggctttctcaatgatggtctacgttttcatatactccaatgaatgtaacagaatgtatctatatggccttgtgcctataaaacaatatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaaccttaccagcttttgttagcttgtgtaatggcttggacttgggggtttattttgccggcttcttaccaagtcagctccccttaaatgcattagccggctgcccgctgtggaccgtctattggtgtgataattatctacgccgtggatgtctactattaatgggttgaagctgcttcaaaccgtctgtttactcagacaattaatgacaatttgacctcaaatcaggtaggactacccgctgaacttaagcatatcaataagcggaggaaaagaaac

SEQ ID NO: 46 - DNA, 658 bp, Psilocybe sp.
  source 1..658: mol_type = genomic DNA
aagtcgtaacaaggtttccgtaggtgaacctgcggaaggatcattattgaataactttggcgtggttgtagctggtcctctcgggggcatgtgctcgcccgtcatctttatgtctccacctgtgcaccttttgtagacgttgaaactggataggagagggacttgtccttcaagttgaaggatttttcggcgctctacgttttcatataccccaaagaatgtaacagaatgtatcttaaggctttatgcctataaactatatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaaccttaccagcttttgttagcttgtgtaatggcttggacttgggggtcttttgccggcttctytcgagatgtcagctccccttaaatgyattagccggctgcccgctgtggaccgtctattggtgtgataattatctacgccgtggaygtctgctctcaatgggttgaagctgcttctaaccgtccgttcattctggacagcacataatgacaatttgacctcaaatca

SEQ ID NO: 47 - DNA, 736 bp, Psilocybe sp.
  source 1..736: mol_type = genomic DNA
gatcattattgaataactttggcgtggttgtagctggtcctctcgggggcatgtgctcgccygtcatctttatatctccacctgtgcaccttttgtagacgttgaaactggataggagagggacttgtccttcaakttaaaggtttttcggcgctctacgttttcatataccccaaagaatgtaacagaatgtatcttatggctttatgcctataaactatatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaaccttaccagcttttgttagcttgtgtaatggcttggacttgggggtmttttgccggcttctctygagatgtcagctccccttaaatgcattagccggctgcccgctgtggaccgtctattggtgtgataattatctacgccgtggacgtctgctctcaatgggttgaagctgcttctaaccgtccgttcattcggacagcacataatgacaatttgacctcaaatcaggtaggactacccgctgaacttaagcatatcaataagcggaggaaaagaaactaacaaggattcccctagtaactgcgagtgaagcgggaaaagctcaaatttaaaatctggcggtct

SEQ ID NO: 48 - DNA, 703 bp, Psilocybe sp.
  source 1..703: mol_type = genomic DNA
gatcattattgaataactttggcgtggttgtagctggtcctctcgggggcatgtgctcgcccgtcatctttatatctccacctgtgcaccttttgtagacgttggaactggataggagagggacttgtccttcaagttgaaggttttttcggcgctctacgttttcatataccccaaagaatgtaacagaatgtatcttatggctttatgcctataaactatatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaaccttaccagcttttgttagcttgtgtaatggcttggacttgggggtattttgccggcttctcttgagatgtcagctccccttaaatgcattagccggctgcccgctgtggaccgtctattggtgtgataattatctacgccgtggacgtctgctctcaatgggttgaagctgcttctaaccgtccgtttattcggacagcacataatgacaatttgacctcaaatcaggtaggactacccgctgaacttaagcatatcaataagcggaggaaaagaaactaacaaggattcccctagtaactgcgagtgaa

SEQ ID NO: 49 - DNA, 701 bp, Psilocybe sp.
  source 1..701: mol_type = genomic DNA
gatcattattgaataactttggcgtggttgtagctggtcctctcgggggcatgtgctcgcccgtcatctttatatctccacctgtgcaccttttgtagacgttgaaactggataggagagggacttgtccttcaagttgaaggatttttcggcgctctacgttttcatataccccaaagaatgtaacagaatgtatcttatggctttatgcctataaactatatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaaccttaccagcttttgttagcttgtgtaatggcttggacttgggggtcttttgccggcttctctcgagatgtcagctccccttaaatgtattagccggctgcccgctgtggaccgtctattggtgtgataattatctacgccgtggacgtctgctctcaatgggttgaagctgcttctaaccgtccgtttattcggacagcacataatgacaatttgacctcaaatcaggtaggactacccgctgaacttaagcatatcaatacggaggaaaagaaactaacaaggattcccctagtaactgcgagtgaa

SEQ ID NO: 50 - DNA, 111 bp, Psilocybe sp.
  source 1..111: mol_type = genomic DNA
ttaattattagggtgcgttggatatcgaagttcggtcagagtaggggcacggcagtggcatggcatgggtaatgttgcaacaatatgatctatttaggtgcgtcattcact

SEQ ID NO: 51 - DNA, 111 bp, Psilocybe sp.
  source 1..111: mol_type = genomic DNA
ttaattattagggtgcgttggatatcgaagttcggtcagagtaggggcaccacagtggcatggcatgggtaatgttgcaacaatatgatctatttaggtgcgtcattcact

SEQ ID NO: 52 - DNA, 111 bp, Psilocybe sp.
  source 1..111: mol_type = genomic DNA
ttaattattagggtgcgttrgatatcgaagttcggtcagagyaggggcacsrcagtggcatggcatggstaatgttgcaacartatgatytatttaggtgcgtcattcact

SEQ ID NO: 53 - DNA, 111 bp, Psilocybe sp.
  source 1..111: mol_type = genomic DNA
ttaattattagggtgcgttagatatcgaagttcggtcagagcaggggcacggcagtggcatggcatggctaatgttgcaacagtatgatttatttaggtgcgtcattcact

SEQ ID NO: 54 - DNA, 111 bp, Psilocybe sp.
  source 1..111: mol_type = genomic DNA
ttaattattagggtgcgttggatatcgaagttcggtcagagtaggggcacnnnngtggcatggcatgggtaatgttgcaacaatatgatctatttaggtgcgtcattcact

SEQ ID NO: 55 - DNA, 111 bp, Psilocybe sp.
  source 1..111: mol_type = genomic DNA
ttaattattagggtgcgttggatatcgaagttcggtcagagtaggggcacsrcagtgggatggcatgggtaatgttgcaacaatatgatctatttaggtgcgtcattcact

SEQ ID NO: 56 - DNA, 111 bp, Psilocybe sp.
  source 1..111: mol_type = genomic DNA
nnnnnnnnnnnnnnnnnttggatatcgaagttcggtcagagtagggnnnnnnnnnnngcatggcatgggtaatgttgcaacaatatgatctatttaggtgcgtcattcact

SEQ ID NO: 57 - DNA, 111 bp, Psilocybe sp.; identical to SEQ ID NO: 51
  source 1..111: mol_type = genomic DNA

SEQ ID NO: 58 - DNA, 111 bp, Psilocybe sp.
  source 1..111: mol_type = genomic DNA
ttaattattagnnnnnnnnnnnnnnnnnnnnnnnnnnnnnnnnnnnnnnnnnnnnnnnnnnnnnnnnnnnnnnnnnnnnnnnnnnnnnnnnnnnnnngtgcgtcattcact

SEQ ID NO: 59 - DNA, 111 bp, Psilocybe sp.
  source 1..111: mol_type = genomic DNA
ttaattattagggtgagttggatatcgaagttcggtcagagtaggggcaccacagtggcatggcatgggtaatgttgcaacaatatgatctatttaggtgcgtcattcact

SEQ ID NO: 60 - DNA, 115 bp, Psilocybe sp.
  source 1..115: mol_type = genomic DNA
ttaatntattagggtgcgttnggantatcgaagttcggtcagagtaggggcaccacagtggcatggcatgggtaatgttgcaacaatatgatctatttaggntgcgtcattcact

SEQ ID NO: 61 - DNA, 116 bp, Psilocybe sp.
  source 1..116: mol_type = genomic DNA
aggaggtgggtcgtaatcgttatccgggatagggtcgccaatcgtgctcggggcttgcgcaaagtaggtacctggaacgttgatgattttgacgattgtcccattgacgggtgcgt

SEQ ID NO: 62 - DNA, 116 bp, Psilocybe sp.
  source 1..116: mol_type = genomic DNA
aggargtgggtcgtaatcgttatccgggatagggtcgccaatcgtgctcggggcttgcgcaaagtaggtacctggaacgttgatgattttgacgattgtcccattgacgggtgcgt

SEQ ID NO: 63 - DNA, 116 bp, Psilocybe sp.
  source 1..116: mol_type = genomic DNA
aggaggtgggtcgtaatcgttatccgggatagggtcgccaatcgtgctcggggcttgcgcaaagtaggtacctggaacgttgatgattttgacgattgtnccattsacgggwgcgt

SEQ ID NO: 64 - DNA, 116 bp, Psilocybe sp.
  source 1..116: mol_type = genomic DNA
aggaggtgggtcgtaatcgttatccgggatagggtcgycaatcgtgctcggggcttgcgcaaagtaggtacctggaacgttgatgattttgacgattgtcccattgacgggtgcgt

SEQ ID NO: 65 - DNA, 22 bp, Psilocybe sp.
  source 1..22: mol_type = genomic DNA
atagggtcgccaatcgtgctcg

SEQ ID NO: 66 - DNA, 19 bp, Psilocybe sp.
  source 1..19: mol_type = genomic DNA
gattgtcccattgacgggt

(19) United States
(12) Patent Application Publication
(10) Pub. No.: US 2026/0085366 A1
(43) Pub. Date: Mar. 26, 2026

(54) PSILOCYBE ASSAY

(71) Applicant: MEDICINAL GENOMICS CORPORATION, Beverly, MA (US)

(72) Inventor: Kevin MCKERNAN, Marblehead, MA (US)

(21) Appl. No.: 19/111,063

(22) PCT Filed: Sep. 11, 2023
(86) PCT No.: PCT/US2023/073835

§ 371 Date: Mar. 12, 2025

Related U.S. Application Data
(60) Provisional application No. 63/375,465, filed on Sep. 13, 2022.

(51) Int. Cl. C12Q 1/6895

(52) U.S. Cl. CPC C12Q 1/6895; C12Q 2600/16

(57) ABSTRACT

A method for assaying putative Psilocybe spores, Psilocybe tissue, or a cultured host organism that expresses Psilocybe genes comprises obtaining a sample including nucleic acids, contacting the sample with primers for amplifying target nucleic acid sequences, amplifying any of the target nucleic acid sequences when present among the nucleic acids to obtain amplicons, and detecting the amplicons upon amplification of the target nucleic acid sequence. In embodiments, the primers include primers for amplifying (i) a bacterial target nucleic acid sequence, (ii) a Psilocybe target nucleic acid sequence, and/or (iii) a fungal target nucleic acid sequence. For instance, the primers include primers for amplifying a Psilocybecubensis target nucleic acid sequence and a target nucleic acid sequence from at least one other mushroom species subject to misidentification as Psilocybecubensis. A kit may include those primers and probes for detecting the amplicons amplified from any of those target nucleic acid sequences.

### CROSS-REFERENCE TO RELATED APPLICATION

This application claims the benefit of U.S. Provisional Application No. 63/375,465, filed on Sep. 13, 2022. The disclosure of the prior application is hereby incorporated by reference herein in its entirety.

### SEQUENCE LISTING

The present application contains a Sequence Listing that has been submitted electronically and is hereby incorporated by reference in its entirety. The electronic Sequence Listing is named Sequence Listing_ST26.xml, which was created on Sep. 11, 2023 and is 65,662 bytes in size.

### BACKGROUND

The Psilocybe genus of mushrooms is well known for the synthesis of valuable psychoactive compounds such as psilocybin (as well as psilocin, baeocystin, aeruginascin, etc.). For instance, psilocybin is especially of commercial interest for various reasons. Of note, psilocybin has been classified as a “breakthrough therapy” for depression by the FDA. FIG. 1 shows that psilocin (which is essentially unphosphorylated psilocybin) is a tryptamine derivative with a structure similar to the mammalian neurotransmitter serotonin. The pharmacological activity of psilocybin (or psilocin) seems to mimic serotonin in the central nervous system, with a high affinity for the 5-HT2A receptor subtype, typical of other hallucinogenic tryptamines.

Psilocybe mushrooms can be grown from Psilocybe spores. The Psilocybe mushrooms may be intended to be edible and thus ingested by humans. Or the Psilocybe mushrooms may be the starting point for further processing to obtain psychoactive compounds such as psilocybin. In any event, there is commercial interest in cultivating Psilocybe mushrooms, or producing psilocybin or psilocybin intermediates in culture, that are safe for ingestion or further processing.

### SUMMARY

In embodiments, a method for assaying (testing) Psilocybe spores, Psilocybe tissue, or a cultured host organism that expresses Psilocybe genes comprises obtaining a sample including nucleic acids, contacting the sample with primers for amplifying target nucleic acid sequences, amplifying any of the target nucleic acid sequences when present among the nucleic acids to obtain amplicons, and detecting the amplicons upon amplification of the target nucleic acid sequence. In embodiments, the primers include primers for amplifying (i) a bacterial target nucleic acid sequence, (ii) a Psilocybe target nucleic acid sequence, and/or (iii) a fungal target nucleic acid sequence. For instance, the primers include primers for amplifying a Psilocybe cubensis target nucleic acid sequence and a target nucleic acid sequence from at least one other mushroom species subject to misidentification as Psilocybe cubensis.

In embodiments, a method for discriminating Psilocybe cubensis from at least one other mushroom species subject to misidentification as Psilocybe cubensis comprises obtaining a sample including nucleic acids from putative Psilocybe cubensis spores or tissue, contacting the sample with primers for amplifying target nucleic acid sequences, amplifying any of the target nucleic acid sequences when present among the nucleic acids to obtain amplicons, and detecting the amplicons upon amplification of the target nucleic acid sequences. In embodiments, the primers include primers for amplifying a Psilocybe cubensis target nucleic acid sequence and a target nucleic acid sequence from the other mushroom species.

For instance, the Psilocybe cubensis target nucleic acid sequence may include an Internal Transcribed Spacer (ITS) target nucleic acid sequence and/or any of a PsiK, PsiM, PsiH, or PsiD target nucleic acid sequence. In that respect, the Psilocybe cubensis target nucleic acid sequence may include at least the Internal Transcribed Spacer (ITS) target nucleic acid sequence. The target nucleic acid sequence from the other mushroom species may include any of an amatoxin target nucleic acid sequence or an Internal Transcribed Spacer (ITS) target nucleic acid sequence. The target nucleic acid sequence from the other mushroom species may include both an amatoxin target nucleic acid sequence and an Internal Transcribed Spacer (ITS) target nucleic acid sequence.

In embodiments, the target nucleic acid sequence from the other mushroom species may include an amatoxin target nucleic acid sequence that is from an amatoxin-producing Galerina species. For instance, the amatoxin target nucleic acid sequence may be from any of Galerina marginata, Galerina venenata, Galerina castaneipes, Galerina fasciculata, Galerina sulcipes, Galerina helvoliceps, Galerina beinrothii, Galerina badipes, Galerina autumnalis, Galerina unicolor, Galerina oregonensis, Galerina physospora, Galerin apatagonica, Galerina jaapii, or Galerina triscopa. In embodiments, the target nucleic acid sequence from the other mushroom species may include an Internal Transcribed Spacer (ITS) target nucleic acid sequence from a Psilocybe species linked to wood lovers paralysis (WLP). For instance, the target nucleic acid sequence from the other mushroom species may include an Internal Transcribed Spacer (ITS) target nucleic acid sequence from any of Psilocybe azurescens, Psilocybe cyanescens, Psilocybe allenii, Psilocybe subaeruginosa, Psilocybe stunzii, Psilocybe pelliculosa, or Psilocybe ovoideocystidiata.

In embodiments, a method of detecting contamination in Psilocybe spores, Psilocybe tissue, or a cultured host organism that expresses Psilocybe genes comprises obtaining a sample including nucleic acids from Psilocybe spores, Psilocybe tissue, or the cultured host organism that expresses Psilocybe genes (hereinafter generically referred to as “Psilocybe sample”), contacting the sample with primers for amplifying target nucleic acid sequences, amplifying any of the target nucleic acid sequences when present among the nucleic acids to obtain amplicons, and detecting the amplicons upon amplification of the target nucleic acid sequence. In embodiments, the primers include primers for amplifying (i) a bacterial target nucleic acid sequence, (ii) a Psilocybe target nucleic acid sequence, and/or (iii) a fungal target nucleic acid sequence.

For instance, the primers may include primers for amplifying a bacterial target nucleic acid sequence and a Psilocybe target nucleic acid sequence. The bacterial target nucleic acid sequence may be any of a 16S target nucleic acid sequence, E. coli target nucleic acid sequence, or Salmonella target nucleic acid sequence. For instance, the bacterial target nucleic acid sequence may include a 16S target nucleic acid sequence. Or the bacterial target nucleic acid sequence may include an E. coli target nucleic acid sequence and/or Salmonella target nucleic acid sequence. The Psilocybe target nucleic acid sequence may be any of a PsiK, PsiM, PsiH, or PsiD target nucleic acid sequence. The Psilocybe target nucleic acid sequence may include a target nucleic acid sequence from each of PsiK and PsiM. The Psilocybe target nucleic acid sequence may include a target nucleic acid sequence from each of PsiK, PsiM, and PsiD.

In embodiments, the Psilocybe sample originates from P. cubensis, P. semilanceata, P. azurescens, P. tampanensis, P. zapotecorum, P. cyanescens, P. caerulescens, P. mexicana, P. caerulipses, P. stuntzii, P. baeocystis, P. bohemica, P. weilii, or P. hoogshagenii. For instance, the Psilocybe sample may originate from P. cubensis.

In embodiments, the Psilocybe genes expressed in the cultured host organism originate from P. cubensis, P. semilanceata, P. azurescens, P. tampanensis, P. zapotecorum, P. cyanescens, P. caerulescens, P. mexicana, P. caerulipses, P. stuntzii, P. baeocystis, P. bohemica, P. weilii, or P. hoogshagenii. For instance, the Psilocybe genes may originate from P. cubensis.

In embodiments, the primers further include primers for amplifying a fungal target nucleic acid sequence. The fungal target nucleic acid sequence may be an Internal Transcribed Spacer (ITS) target nucleic acid sequence.

In embodiments, detection of the amplicons includes quantification of the amplicons. For instance, quantification of amplicons amplified from the bacterial target nucleic acid sequence may be a measure of bacterial contamination. Further, quantification of amplicons amplified from the fungal target nucleic acid sequence may be a measure of fungal contamination. But quantification of amplicons amplified from the Psilocybe target nucleic acid sequence may be a confirmatory measure of presence of Psilocybe spores in the sample.

In embodiments, a polymerase chain reaction (PCR) is performed to amplify the target nucleic acid sequences. For instance, the PCR may be a real-time polymerase chain reaction (qPCR). And multiplex amplification may be performed to amplify the target nucleic acid sequences.

In embodiments, probes are hybridized to the amplicons to detect the amplicons. The probes may include a fluorescent label. And the probes may have different fluorescent labels based on different amplicon sequences to which the probes hybridize. Further, the probes may have a probe structure that includes a fluorophore and a quencher. Exemplary fluorophores include 56-FAM, 56-ROXN, 5TEX615, and 5HEX. Exemplary quenchers include 3IABkFQ, 3IAbRQSp, and ZEN.

In embodiments, the primers include a primer having a sequence that is at least 70%, 75%, 80%, 85%, 90%, or 95% identical to any one of SEQ ID NOS: 12-15, 18, 19, 21, 22, 24, 25, 27, 28, 30, 31, 33-38, 42, or 43. In embodiments, the probes include a probe having a sequence that is at least 70%, 75%, 80%, 85%, 90%, or 95% identical to any one of SEQ ID NOS: 16, 17, 20, 23, 26, 29, 32, 39-41, or 44. Any of the primers and/or probes may hybridize to regions that have less than 1% genomic variation in a sequence alignment performed for multiple Psilocybe strains.

In embodiments, the target nucleic acid sequences include a target nucleic acid sequence that is at least 85%, 86%, 87%, 88%, 89%, 90%, 91%, 92%, 93%, 94%, 95%, 96%, 97%, 98%, or 99% identical to any one of SEQ ID NOS: 1-3 or 7. In embodiments, the bacterial and Psilocybe target nucleic acid sequences include a target nucleic acid sequence that is at least 85%, 86%, 87%, 88%, 89%, 90%, 91%, 92%, 93%, 94%, 95%, 96%, 97%, 98%, or 99% identical to any one of SEQ ID NOS: 4-6 or 8-10. In embodiments, the fungal target nucleic acid sequence is at least 85%, 86%, 87%, 88%, 89%, 90%, 91%, 92%, 93%, 94%, 95%, 96%, 97%, 98%, or 99% identical to SEQ ID NO: 11.

In embodiments, a kit for assaying (testing) Psilocybe spores, Psilocybe tissue, or a cultured host organism that expresses Psilocybe genes comprises (i) primers for amplifying a bacterial target nucleic acid sequence, a Psilocybe target nucleic acid sequence, and/or a fungal target nucleic acid sequence, and (ii) probes for detecting the amplicons amplified from any of those target nucleic acid sequences. For instance, the primers may include primers for amplifying a Psilocybe cubensis target nucleic acid sequence and a target nucleic acid sequence from at least one other mushroom species subject to misidentification as Psilocybe cubensis.

In embodiments, a kit for discriminating Psilocybe cubensis from at least one other mushroom species subject to misidentification as Psilocybe cubensis comprises (i) primers for amplifying a Psilocybe cubensis target nucleic acid sequence and a target nucleic acid sequence from the other mushroom species and (ii) probes for detecting amplicons of the Psilocybe cubensis target nucleic acid sequence and the target nucleic acid sequence from the other mushroom species. The probes may include a fluorescent label.

In embodiments, the kit further comprises reagents including any of a lysis buffer, magnetic beads, a binding buffer, a wash solution, an elution solution, a DNA polymerase, or dNTPs. The kit may also further comprise instructions for performing an assay (or a test) to discriminate Psilocybe cubensis from the other mushroom species.

In embodiments, the Psilocybe cubensis target nucleic acid sequence may include an Internal Transcribed Spacer (ITS) target nucleic acid sequence and/or any of a PsiK, PsiM, PsiH, or PsiD target nucleic acid sequence. For instance, the Psilocybe cubensis target nucleic acid sequence may include at least the Internal Transcribed Spacer (ITS) target nucleic acid sequence. The target nucleic acid sequence from the other mushroom species may include any of an amatoxin target nucleic acid sequence or an Internal Transcribed Spacer (ITS) target nucleic acid sequence. The target nucleic acid sequence from the other mushroom species may include both an amatoxin target nucleic acid sequence and an Internal Transcribed Spacer (ITS) target nucleic acid sequence.

In embodiments, the target nucleic acid sequence from the other mushroom species may include an amatoxin target nucleic acid sequence that is from an amatoxin-producing Galerina species. For instance, the amatoxin target nucleic acid sequence may be from any of Galerina marginata, Galerina venenata, Galerina castaneipes, Galerina fasciculata, Galerina sulcipes, Galerina helvoliceps, Galerina beinrothii, Galerina badipes, Galerina autumnalis, Galerina unicolor, Galerina oregonensis, Galerina physospora, Galerina patagonica, Galerina jaapii, or Galerina triscopa. In embodiments, the target nucleic acid sequence from the other mushroom species may include an Internal Transcribed Spacer (ITS) target nucleic acid sequence from a Psilocybe species linked to wood lovers paralysis (WLP). For instance, the target nucleic acid sequence from the other mushroom species may include an Internal Transcribed Spacer (ITS) target nucleic acid sequence from any of Psilocybe azurescens, Psilocybe cyanescens, Psilocybe allenii, Psilocybe subaeruginosa, Psilocybe stunzii, Psilocybe pelliculosa, or Psilocybe ovoideocystidiata.

In embodiments, a kit for detecting contamination in a Psilocybe sample comprises (i) primers for amplifying a bacterial target nucleic acid sequence, a Psilocybe target nucleic acid sequence, and/or a fungal target nucleic acid sequence, and (ii) probes for detecting the amplicons amplified from any of those target nucleic acid sequences. For instance, the kit may comprise primers for amplifying a bacterial target nucleic acid sequence and a Psilocybe target nucleic acid sequence and probes for detecting amplicons of the bacterial target nucleic acid sequence and the Psilocybe target nucleic acid sequence. The probes may include a fluorescent label.

In embodiments, the kit further comprises reagents including any of a lysis buffer, magnetic beads, a binding buffer, a wash solution, an elution solution, a DNA polymerase, or dNTPs. The kit may also further comprise instructions for performing an assay (or a test) to detect contamination in a Psilocybe sample.

In embodiments, the bacterial target nucleic acid sequence is any of a 16S target nucleic acid sequence, E. coli target nucleic acid sequence, or Salmonella target nucleic acid sequence nucleic acid sequence and the Psilocybe target nucleic acid sequence is any of a PsiK, PsiM, PsiH, or PsiD target nucleic acid sequence. For instance, the bacterial target nucleic acid sequence may include a 16S target nucleic acid sequence. Or the bacterial target nucleic acid sequence may include an E. coli target nucleic acid sequence and/or Salmonella target nucleic acid sequence. The Psilocybe target nucleic acid sequence may be any combination of PsiK, PsiM, PsiH, or PsiD target nucleic acid sequences. For example, the Psilocybe target nucleic acid sequence may include a target nucleic acid sequence from each of PsiK and PsiM or may include a target nucleic acid sequence from each of PsiK, PsiM, and PsiD.

In embodiments, the kit comprises primers for amplifying a fungal target nucleic acid sequence and a probe for detecting amplicons of the fungal target nucleic acid sequence. The fungal target nucleic acid sequence may be an Internal Transcribed Spacer (ITS) target nucleic acid sequence.

In embodiments, the probes have different fluorescent labels based on different amplicons sequences to which the probes hybridize. The probes may have a probe structure that includes a fluorophore and a quencher.

In embodiments, the primers include a primer having a sequence that is at least 70%, 75%, 80%, 85%, 90%, or 95% identical to any one of SEQ ID NOS: 12-15, 18, 19, 21, 22, 24, 25, 27, 28, 30, 31, 33-38, 42, or 43. In embodiments, the probes include a probe having a sequence that is at least 70%, 75%, 80%, 85%, 90%, or 95% identical to any one of SEQ ID NOS: 16, 17, 20, 23, 26, 29, 32, 39-41, or 44. Any of the primers and/or probes may hybridize to regions that have less than 1% genomic variation in a sequence alignment performed for multiple Psilocybe strains.

In embodiments, the target nucleic acid sequences include a target nucleic acid sequence that is at least 85%, 86%, 87%, 88%, 89%, 90%, 91%, 92%, 93%, 94%, 95%, 96%, 97%, 98%, or 99% identical to any one of SEQ ID NOS: 1-3 or 7. In embodiments, the bacterial and Psilocybe target nucleic acid sequences include a target nucleic acid sequence that is at least 85%, 86%, 87%, 88%, 89%, 90%, 91%, 92%, 93%, 94%, 95%, 96%, 97%, 98%, or 99% identical to any one of SEQ ID NOS: 4-6 or 8-10. In embodiments, the fungal target nucleic acid sequence is at least 85%, 86%, 87%, 88%, 89%, 90%, 91%, 92%, 93%, 94%, 95%, 96%, 97%, 98%, or 99% identical to SEQ ID NO: 11.

### BRIEF DESCRIPTION OF THE FIGURES

Those of skill in the art will understand that the figures described below are for illustrative purposes only. The figures are not intended to be limiting in any way.

FIG. 1 shows that psilocin (which is essentially unphosphorylated psilocybin) is a tryptamine derivative with a structure similar to serotonin.

FIG. 2 shows examples that illustrate that P. cubensis is difficult to visually differentiate from Psilocybe species linked to (associated with) wood lovers paralysis (WLP) or toxic mushrooms like Galerina marginata.

FIG. 3 shows enzymatic synthesis of psilocybin from L-tryptophan.

FIG. 4 shows an example of a syringe holding spores after being vortexed.

FIG. 5 shows an example of spores concentrated into 100 μl in a tube after centrifugation of 1 ml of the spores.

FIG. 6 shows an exemplary pipette tip with dark spores in which the presence of the spores can be confirmed by visual detection.

FIG. 7 shows examples of boiled spores.

FIG. 8 shows spore samples prepared for qPCR.

FIG. 9 shows qPCR results from assaying P. cubensis spores and non-P. cubensis spores.

FIG. 10 also shows qPCR results from assaying P. cubensis spores and non-P. cubensis spores.

FIG. 11 shows qPCR results from assaying amatoxin and Psilocybe species linked to wood lovers paralysis (WLP).

FIG. 12 shows an example of syringes holding an aqueous solution of P. cubensis spores.

FIG. 13 shows qPCR results from assaying P. cubensis spores having bacterial contamination.

FIG. 14 shows qPCR results from assaying P. cubensis spores that are not contaminated as compared to the qPCR results shown by FIG. 13.

FIG. 15 shows qPCR results from assaying P. cubensis spores from various P. cubensis for PsiK and PsiM genes.

FIG. 16 shows exemplary sequence alignments of Internal Transcribed Spacer (ITS) nucleic acid sequences from Psilocybe species linked to wood lovers paralysis (WLP). SEQ ID NOS: 45-49 are set forth in FIG. 16.

FIG. 17 shows a sequence alignment indicating polymorphic PsiD regions between different Psilocybe strains/species/samples. SEQ ID NOS: 50-60 are set forth in FIG. 17.

FIG. 18 shows a sequence alignment indicating conserved PsiD regions between different Psilocybe strains/species/samples. SEQ ID NOS: 61-66 are set forth in FIG. 18.

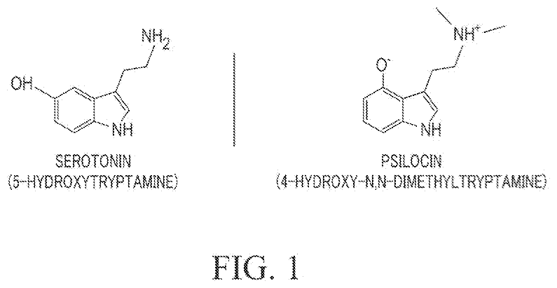

### DETAILED DESCRIPTION OF EMBODIMENTS

There is commercial interest in cultivating Psilocybe mushrooms, or producing psilocybin or psilocybin intermediates from a cultured host organism that expresses Psilocybe genes, that are safe for ingestions or further processing. Thus, embodiments are directed to methods for assaying (testing) putative Psilocybe spores, putative Psilocybe tissue, or a cultured host organism that expresses Psilocybe genes that each comprise obtaining a sample including nucleic acids, contacting the sample with primers for amplifying target nucleic acid sequences, amplifying any of the target nucleic acid sequences when present among the nucleic acids to obtain amplicons, and detecting the amplicons upon amplification of the target nucleic acid sequence.

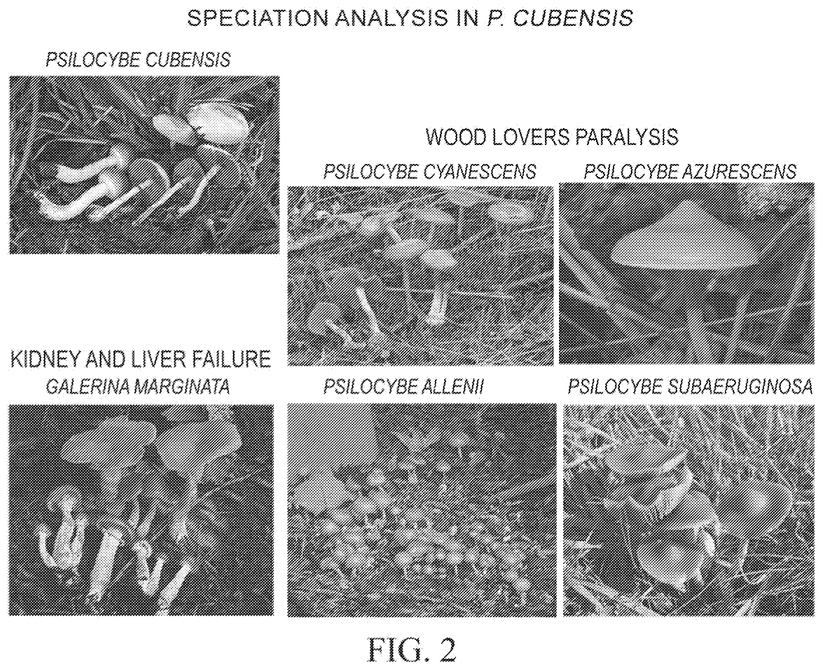

Poisonous mushrooms are subject to misidentification as Psilocybe cubensis, which can lead to harm or even death. FIG. 2 shows examples that illustrate that P. cubensis is difficult to visually differentiate from other Psilocybe species linked to (associated with) wood lovers paralysis (WLP) or toxic mushrooms like Galerina marginata.

Toxic mushroom species such as Galerina marginata produce an amatoxin that can be deadly upon ingestion. Amatoxin is the collective name of a subgroup of at least nine related toxic compounds found in three genera of poisonous mushrooms (Amanita, Galerina and Lepiota) and one species (Conocybe filaris) of the genus Conocybe. Galerina marginata (known colloquially as funeral bell, deadly skullcap, autumn skullcap, or deadly galerina) is a species of extremely poisonous mushroom-forming fungus in the family Hymenogastraceae of the order Agaricales. It contains the same deadly amatoxins found in the death cap (Amanitaphalloides). Ingestion in toxic amounts causes severe liver damage with, for example, vomiting, diarrhea, hypothermia, and/or eventual death if not treated rapidly.

Other members of the Galerina genus are also known to produce an amatoxin. Known Galerina amatoxin-producers are included in the Naucoriopsis subgenus (or clade) and include Galerina marginata, Galerina venenata, Galerina castaneipes, Galerinafasciculata, Galerina sulcipes, Galerina helvoliceps, Galerina beinrothii, Galerina badipes, Galerina autumnalis, Galerina unicolor, Galerina oregonensis, Galerina physospora, Galerina patagonica, Galerina jaapii, or Galerina triscopa.

Of note, Galerina marginata is widespread in the Northern Hemisphere, including Europe, North America, and Asia, and has also been found in Australia. It is a wood-rotting fungus that grows predominantly on decaying conifer wood. The fruit bodies of the mushroom have brown to yellow-brown caps that fade in color when drying. The gills are brownish and give a rusty spore print. A well-defined membranous ring is typically seen on the stems of young specimens but often disappears with age. In older fruit bodies, the caps are flatter and the gills and stems browner. Before 2001, the species G. autumnalis, G. oregonensis, G. unicolor, and G. venenata were thought to be distinct from G. marginata due to differences in habitat and the viscidity of their caps, but phylogenetic analysis showed that they are all the same species. Galerina marginata may be easily confused with several edible species including Psilocybe cubensis.

Other poisonous mushroom species includes those linked to (associated with) with wood lovers paralysis (WLP). WLP refers to a temporary state of muscle weakness and/or paralysis that begins several hours after consuming certain types of magic mushrooms (aka psilocybin mushrooms or psychedelic mushrooms). The phenomenon appears to occur only after ingesting certain mushroom species that grow on wood, hence the name wood lovers paralysis. WLP is reported most often for the species Psilocybe cyanescens and Psilocybe azurescens. It has also been reported for P. allenii, P. stunzii, P. subaeruginosa, P. pelliculosa, and P. ovoideocystidiata (among others).

In embodiments, a method for discriminating Psilocybe cubensis from at least one other mushroom species subject to misidentification as Psilocybe cubensis comprises obtaining a sample including nucleic acids from putative Psilocybe cubensis spores or tissue, contacting the sample with primers for amplifying target nucleic acid sequences, amplifying any of the target nucleic acid sequences when present among the nucleic acids to obtain amplicons, and detecting the amplicons upon amplification of the target nucleic acid sequences. In embodiments, the primers include primers for amplifying a Psilocybe cubensis target nucleic acid sequence and a target nucleic acid sequence from the other mushroom species.

The method may be used for assaying (i.e., testing) a putative Psilocybe cubensis sample to confirm whether or not it is indeed Psilocybe cubensis. If it is not, the method may further identify it as a mushroom that produces an amatoxin or a Psilocybe species linked to wood lovers paralysis (WLP). In any event, samples determined to be obtained from potentially useless and/or harmful mushrooms can be discarded and/or destroyed. In that way, misidentification of other mushroom species as Psilocybe cubensis can be avoided. Embodiments of methods for discriminating Psilocybe cubensis from other mushroom species subject to misidentification as Psilocybe cubensis are described in greater detail below.

Further, bacterial contamination and/or non-Psilocybe fungal contamination can negatively affect the growth, productivity, yield, and/or safety of Psilocybe mushrooms or the cultured host organism. Antibiotics can often be added to reduce bacterial contamination, but not all fungi or potential host organisms tolerate antibiotics and not all users of psilocybin or Psilocybe mushrooms tolerate residual antibiotics.

As a result, there exists a desire to screen Psilocybe tissue or a cultured host organism expressing Psilocybe genes for bacterial contamination and/or non-target fungal contamination. And there exists a desire to screen Psilocybe spores for bacterial contamination and/or non-target fungal contamination prior to inoculation. This is particularly important in the growth of edible mushrooms like Psilocybe where it is preferred to grow these mushrooms in the absence of antibiotics to avoid antibiotic contamination of edible mushrooms. The spawning stage of mycelium growth is particularly sensitive to bacterial contamination as the process can take 2-4 weeks for fully mature mycelium to grow. Injecting bacterially contaminated spores into spawning grains or medium will result in contaminated growth and failed Psilocybe growth. Further, assaying/testing Psilocybe spores may prevent misidentification of spores from poisonous mushrooms like Galerina marginata or Cortinarius as being Psilocybe spores.

In embodiments, a method of detecting contamination in a Psilocybe sample thus comprises obtaining a sample including nucleic acids from Psilocybe spores, Psilocybe tissue, or a cultured host organism that expresses Psilocybe genes, contacting the sample with primers for amplifying target nucleic acid sequences, amplifying any of the target nucleic acid sequences when present among the nucleic acids to obtain amplicons, and detecting the amplicons upon amplification of the target nucleic acid sequence. In embodiments, the primers include primers for amplifying (i) a bacterial target nucleic acid sequence, (ii) a Psilocybe target nucleic acid sequence, and/or (iii) a fungal target nucleic acid sequence.

The method may be used for assaying (i.e., testing) a Psilocybe sample for contamination from bacteria and other fungal (e.g., yeast and mold) sources. In the case of Psilocybe spores, the contamination may be quantified (i.e., measured) so as to determine whether the Psilocybe spores are suitable for growing Psilocybe mushrooms. In other words, the method may be used to screen Psilocybe spores, Psilocybe tissue, or a cultured host organism that expresses Psilocybe genes for contamination. Contaminated samples may then be discarded in favor of samples with less contamination (e.g., without significant and/or detectable contamination). Embodiments of methods for detecting contamination in a Psilocybe sample are described in greater detail below.

### Psilocybe Sample

In embodiments, a sample including nucleic acids is obtained. For instance, the sample may be from putative Psilocybe cubensis spores or tissue. In embodiments, the sample is subject to assaying/testing so as to discriminate Psilocybe cubensis from at least one other mushroom species subject to misidentification as Psilocybe cubensis. In embodiments, the other mushroom species subject to misidentification may be (i) a mushroom species that produces an amatoxin such as Galerina marginata, Galerina venenata, Galerina castaneipes, Galerina fasciculata, Galerina sulcipes, Galerina helvoliceps, Galerina beinrothii, Galerina badipes, Galerina autumnalis, Galerina unicolor, Galerina oregonensis, Galerina physospora, Galerina patagonica, Galerina jaapii, or Galerina triscopa or (ii) a Psilocybe species linked to wood lovers paralysis (WLP) such as Psilocybe azurescens, Psilocybe cyanescens, Psilocybe allenii, Psilocybe subaeruginosa, Psilocybe stunzii, Psilocybe pelliculosa, or Psilocybe ovoideocystidiata.

In embodiments, the Psilocybe sample is subject to assaying/testing for contamination. The Psilocybe spores or tissue may originate from P. cubensis, P. semilanceata, P. azurescens, P. tampanensis, P. zapotecorum, P. cyanescens, P. caerulescens, P. mexicana, P. caerulipses, P. stuntzii, P. baeocystis, P. bohemica, P. weilii, or P. hoogshagenii. Likewise, the Psilocybe genes expressed in the cultured host organism may originate from P. cubensis, P. semilanceata, P. azurescens, P. tampanensis, P. zapotecorum, P. cyanescens, P. caerulescens, P. mexicana, P. caerulipses, P. stuntzii, P. baeocystis, P. bohemica, P. weilii, or P. hoogshagenii. For instance, the Psilocybe sample may originate from P. cubensis. In embodiments, the Psilocybe spores, tissue, or genes originate from P. cubensis.

Psilocybe spores such as P. cubensis spores (including putative spores) may be obtained from commercial sources (e.g., sporeworks.com, premiumspores.com, mushroom.com, inoculatetheworld.com, etc.) or may be otherwise prepared. Spores may be provided in a syringe (e.g., containing an aqueous suspension/solution of the spores). To assay/test for discrimination or contamination purposes, a sample is obtained of the Psilocybe spores (e.g., a sample of an aqueous suspension/solution of the spores).

Psilocybe tissue such as P. cubensis tissue (including putative tissue) may be obtained from any part of the Psilocybe fungus, such as the mycelium or the fruiting body (which includes the trama, the hymenium, and the pileipellis). The tissue can be fresh (hydrated) or dried. The tissue could be an intact mushroom, one or more tissue layers of the mushroom (in combination or isolated), or a cellular sample. To assay/test for discrimination or contamination, a sample is obtained of the Psilocybe tissue (e.g., a sample of an aqueous suspension/solution of the cells or homogenized tissue).

A host organism that expresses Psilocybe genes can be used to produce psilocybin or psilocybin intermediates in culture. This has the potential to be a more environmentally benign process with higher throughput. For example, engineered psilocybin biosynthesis genes can be expressed in a recombinant host organism.

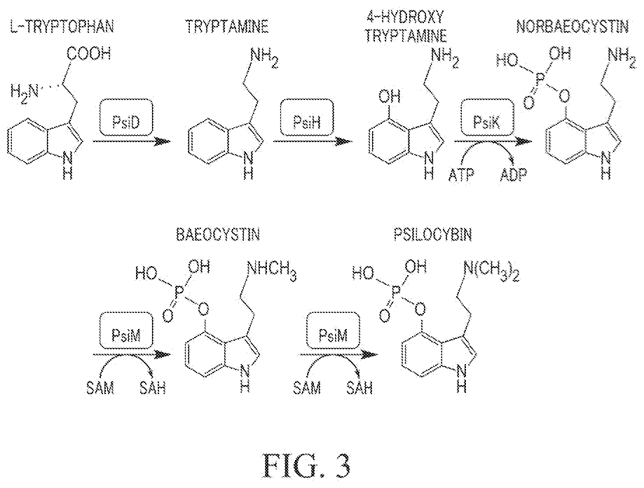

FIG. 3 shows enzymatic synthesis of psilocybin from L-tryptophan. The biosynthetic production of psilocybin starts with L-tryptophan, which is converted into tryptamine by tryptophan decarboxylase. Tryptamine is next converted into 4-hydroxytryptamine by a cytochrome P450 containing monooxygenase (PcPsiH). Cytochrome P450 enzymes are characterized by their dependency on a cytochrome P450 reductase (CPR), which facilitates electron transfer between NADPH and cytochrome P450 enzymes. 4-hydroxytryptamine is next converted into norbaeocystin by a 4-hydroxytryptamine kinase encoded by PcPsiK, which facilitates the 4-O-phosphorylation reaction. Finally, an N-methyltransferase encoded by PcPsiM mediates the iterative methyl transfer of norbaeocystin to baeocystin then to psilocybin.

The recombinant host can be modified to include genes encoding the enzymes involved in the psilocybin biosynthesis pathway. Alternatively, endogenous pathways of the recombinant host can be modified to produce high purity psilocybin and/or intermediates of psilocybin. For example, production of psilocybin can be increased by promoting expression of CPR in the host organism.

Recombinant host organisms include, for example, E. co/i, Schizosaccharomyces cerevisiae, Schizosaccharomyces japonicus, Schizosaccharomyces pombe, Schizosaccharomyces cryophilus, Saccharomyces cerevisiae, Kluyveromyces lactis, Kluyveromyces dobzhanskii, and Yarrowia lipolytica. Genes that can be inserted or engineered into the recombinant host include PsiK, PsiM, PsiH, and PsiD, and genes encoding other enzymes involved in the psilocybin biosynthesis pathway. The engineered enzymes act on substrates in the psilocybin biosynthetic pathway (e.g., L-tryptophan and/or 4-hydroxy-L-tryptophan) to produce intermediates of psilocybin and psilocybin itself. See, e.g., Gibbons Jr., Bioengineered 12:8863 (2021); Milne, Metabolic Engineering 60:25 (2020); U.S. 2021/0147888 (the disclosures of which are incorporated herein).

### Obtaining Nucleic Acids

In embodiments, a sample including nucleic acids is obtained from the Psilocybe sample (including a P. cubensis or putative P. cubensis sample). The nucleic acids may be DNA and/or RNA. In that respect, nucleic acids are polymers of nucleotides (e.g., ribonucleotides and deoxyribonucleotides, both natural and non-natural, including adenine (A), cytosine (C), guanine (G), thymine (T), and uracil (U)) such polymers being DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages.

Nucleic acids will typically include naturally occurring bases (adenine (A), cytosine (C), guanine (G), thymine (T), and/or uracil (U)) but it is possible that they contain non-natural bases in embodiments. (The example of non-natural bases include those described in, e.g., Seela et al. (1999) Helv. Chim. Acta 82:1640. Certain bases used in nucleotide analogs act as melting temperature (T) modifiers. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytidine; 5-fluorocytidine; 5-chlorocytidine; 5-iodocytidine; 5-bromocytidine; 5-methylcytidine; 5-propynylcytidine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.)

In embodiments, the nucleic acids include genomic DNA. In other embodiments, the nucleic acids will include mRNA or cDNA produced from the mRNA for purposes of detecting gene expression as opposed to genomic DNA per se. As is well known, the genetic framework of an organism is encoded in the double-stranded sequence of nucleotide bases in the DNA which is contained in the cells of the organism. The genetic content of a particular segment of DNA, or gene, is manifested only upon production of the gene product (RNA or protein) encoded by the gene. To produce a gene product, a complementary copy of one strand of the DNA double helix (the “coding” strand) is produced by polymerase enzymes, resulting in a specific sequence of RNA. When this particular type of RNA contains the genetic message from the DNA for production of a protein, it is called messenger RNA (mRNA).

A common approach to the study of gene expression is the production of complementary DNA (cDNA). In this technique, the RNA (e.g., mRNA) molecules from an organism are isolated from an extract of the cells (or tissues) of the organism. From these purified mRNA molecules, cDNA copies may be made using the enzyme reverse transcriptase (RT) or DNA polymerases having RT activity, which results in the production of single-stranded cDNA molecules.

Reverse transcriptases are a class of polymerases characterized as RNA dependent DNA polymerases. Known reverse transcriptases require a primer to synthesize a DNA transcript from an RNA template. Avian myoblastosis virus (AMV) reverse transcriptase was the first widely used RNA dependent DNA polymerase (Verma, Biochem. Biophys. Acta 473:1(1977)). The enzyme has 5′-3′ RNA directed DNA polymerase activity, 5′-3′ DNA directed DNA polymerase activity, and RNase H activity. RNase H is a processive 5′ and 3′ ribonuclease specific for the RNA strand for RNA DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365 2372 (1983). Another reverse transcriptase which is used extensively in molecular biology is reverse transcriptase originating from Moloney murine leukemia virus (M-MLV). See, e.g., Gerard, G. R., DNA 5:271 279 (1986) and Kotewicz, M. L., et al., Gene 35:249 258 (1985). M-MLV reverse transcriptase substantially lacking in RNase H activity has also been described. See, e.g., U.S. Pat. No. 5,244,797.

Conventional protocols for obtaining DNA or RNA from cells are described in the literature. See, e.g., Chapter 2 (DNA) and Chapter 4 (RNA) of F. Ausubel et al., eds., Current Protocols in Molecular Biology, Wiley-Interscience, New York (1993). Conventional DNA isolation protocols generally entail suspending the cells in a solution and using enzymes and/or chemicals to lyse the cells, thereby releasing the DNA contained within the cells into the resulting lysate solution. The enzymes and/or chemicals may be provided in a lysis buffer. For isolation of RNA, the conventional lysis and solubilization procedures include measures for inhibition of ribonucleases and contaminants to be separated from the RNA including DNA.

Any one of a number of different known methods for lysing or disrupting cells to release nucleic acid materials contained therein are suitable for use in producing a medium from cells. For example, in order to cause a cell with a relatively hard cell wall, such as a fungus cell, to release the nucleic acid material contained therein one may need to use harsher treatments such as potent proteases and mechanical shearing with a homogenizer or disruption with sound waves using a sonicator. Once the nucleic acid material is released from cells lysed or disrupted, cellular debris can be removed using a number of different known techniques or combination of techniques. The solution of lysed or disrupted cells may be centrifuged to remove particulate cell debris. The supernatant may then be further processed by adding a second solution to the supernatant which causes a precipitate of additional other material to form, and then removing the precipitate from the resulting solution by centrifugation.

Many conventional protocols generally entail use of phenol or an organic solvent mixture containing phenol and chloroform to extract additional cellular material such as proteins and lipids from a conventional lysate solution produced as described above. The phenol/chloroform extraction step is generally followed by precipitation of the nucleic acid material remaining in the extracted aqueous phase by adding ethanol to that aqueous phase. The precipitate is typically removed from the solution by centrifugation, and the resulting pellet of precipitate is allowed to dry before being resuspended in water or a buffer solution for further processing or analysis.

Glass particles, silica particles, silica gel, and mixtures thereof have been configured in various different forms to produce matrices capable of reversibly binding nucleic acid materials when placed in contact with a medium containing such materials in the presence of chaotropic agents. Such matrices are designed to remain bound to the nucleic acid material while the matrix is exposed to an external force such as centrifugation or vacuum filtration to separate the matrix and nucleic acid material bound thereto from the remaining media components. The nucleic acid material is then eluted from the matrix by exposing the matrix to an elution solution, such as water or an elution buffer. Numerous commercial sources offer silica-based matrices designed for use in centrifugation and/or filtration isolation systems. See, e.g., Wizard™ DNA purification systems line of products from Promega Corporation (Madison, Wisconsin, USA); or the QiaPrep™ line of DNA isolation systems from Qiagen Corp. (Chatsworth, California, USA).

Magnetically responsive particles (“magnetic particles”) and methods for using magnetic particles have been developed for the isolation of nucleic acid materials. Several different types of magnetic particles designed for use in nucleic acid isolation are described in the literature, and many of those types of particles are available from commercial sources. Such magnetic particles generally fall into either of two categories, those designed to reversibly bind nucleic acid materials directly, and those designed to do so through at least one intermediary label.

The magnetic particles designed to bind nucleic acid materials indirectly are generally used to isolate a specific nucleic acid material, such as mRNA, according to the following basic isolation procedure. First, a medium containing a nucleic acid material is placed in contact with a label capable of binding to the nucleic acid material of interest. For example, one such commonly employed label, biotinylated oligonucleotide deoxythymidine (oligo-dT), forms hydrogen bonds with the poly-adenosine tails of mRNA molecules in a medium. Each label so employed is designed to bind with a magnetically responsive particle, when placed into contact with the particle under the proper binding conditions. For example, the biotin end of a biotinylated oligo-dT/mRNA complex is capable of binding to streptavidin moieties on the surface of a streptavidin coated magnetically responsive particle. Several different commercial sources are available for streptavidin magnetic particles and reagents designed to be used in mRNA isolation using biotinylated oligo-dT as described above. See, e.g., PolyATtract™ Series 9600™ mRNA Isolation System from Promega Corporation; or the ProActive™ line of streptavidin coated microsphere particles from Bangs Laboratories (Carmel, Indiana, USA).

Magnetic particles and label systems have also been developed which are capable of indirectly binding and isolating other types of nucleic acids, such as double-stranded and single-stranded PCR templates. See, e.g., BioMag™ superparamagnetic particles from Advanced Magnetics, Inc. (Cambridge, Massachusetts, USA). Indirect binding magnetic separation systems for nucleic acid isolation or separation all require at least three components, i.e. magnetic particles, a label, and a medium containing the nucleic acid material of interest. The label/nucleic acid binding reaction and label/particle binding reaction often require different solution and/or temperature reaction conditions from one another.

Other types of magnetic particles have also been developed for use in the direct binding and isolation of biological materials, particularly nucleic acids. One such particle type is a magnetically responsive glass bead that may be of a controlled pore size. See, e.g., Magnetic Porous Glass (MPG) particles from CPG, Inc. (Lincoln Park, New Jersey, USA); or porous magnetic glass particles described in U.S. Pat. Nos. 4,395,271; 4,233,169; or 4,297,337. Another type of magnetically responsive particles designed for use in direct binding and isolation of biological materials, particularly nucleic acids, are particles comprised of agarose embedded with smaller ferromagnetic particles and coated with glass. See, e.g., U.S. Pat. No. 5,395,498.

The external magnetic field may be suitably generated using any one of a number of different known means. For example, one can position a magnet on the outer surface of a container of a solution containing the particles, causing the particles to migrate through the solution and collect on the inner surface of the container adjacent to the magnet. The magnet can then be held in position on the outer surface of the container such that the particles are held in the container by the magnetic field generated by the magnet, while the solution is decanted out of the container and discarded. A second solution can then be added to the container, and the magnet removed so that the particles migrate into the second solution. Alternatively, a magnetizable probe could be inserted into the solution and the probe magnetized, such that the particles deposit on the end of the probe immersed in the solution. The probe could then be removed from the solution, while remaining magnetized, immersed into a second solution, and the magnetic field discontinued permitting the particles go into the second solution. Commercial sources exist for magnets designed to be used in both types of magnetic removal and transfer techniques described in general terms above. See, e.g., MagneSphere™ Technology Magnetic Separation Stand or the PolyATract™ Series 960™ Multi-Magnet, both available from Promega Corporation; Magnetight Separation Stand (Novagen, Madison, Wisconsin, USA); or Dynal Magnetic Particle Concentrator (Dynal, Oslo, Norway).

The complex of nucleic acids with the particles that is removed from the medium may be washed at least once by being rinsed in a wash solution. The wash solution used in this additional step of the method may comprise a solution capable of removing contaminants from the silica magnetic particle. The wash solution may comprise a salt and a solvent, such as an alcohol. The concentration of alcohol in embodiments of the wash solution may be at least 30% by volume, at least 40% by volume, or at least 50% by volume. The alcohol so used may be ethanol or isopropanol. The salt may be in the form of a buffer, such as in the form of an acetate buffer. The concentration of salt in the wash solution is sufficiently high to ensure the nucleic acid material is not eluted from the silica magnetic particles during the wash step(s).

The nucleic acid material may then be eluted from the particles by exposing the complex to an elution solution. The elution solution may be an aqueous solution of low ionic strength, such as water or a low ionic strength buffer at about a pH at which the nucleic acid material is stable and substantially intact. Any aqueous solution with an ionic strength at or lower than TE buffer (i.e. 10 mM Tris-HCl, 1 mM ethylenediamine-tetraacetic acid (EDTA), pH 8.0) is suitable for use in the elution steps. The elution solution may be buffered to a pH between about 6.5 and 8.5 or buffered to a pH between about 7.0 and 8.0. TE Buffer and distilled or deionized water are exemplary elution solutions for use. The low ionic strength of such an elution solution ensures the nucleic acid material is released from the particle. Other elution solutions suitable for use in the methods will be readily apparent to one skilled in this art.

### AMPLIFICATION AND DETECTION

In embodiments, the method includes contacting the sample with primers for amplifying target nucleic acid sequences, amplifying any of the target nucleic acid sequences when present among the nucleic acids to obtain amplicons, and detecting the amplicons upon amplification of the target nucleic acid sequences. In that respect, Psilocybe cubensis and at least one other mushroom species subject to misidentification as Psilocybe cubensis may be discriminated. Or bacterial contamination and/or fungal (e.g., yeast and mold) contamination may be detected and/or quantified (i.e., measured) with respect to a Psilocybe sample.

Amplification refers to any chemical reaction, including an enzymatic reaction, which results in increased copies of a template nucleic acid sequence or results in transcription of a template nucleic acid. Such nucleic acid amplification reactions include non-isothermal amplification methods such as polymerase chain reaction (PCR), particularly quantitative/real-time PCR or isothermal amplification methods such as NASBA (nucleic acids sequence based amplification), TMA (Transcription mediated amplification), 3SR (self-sustained sequence amplification), SDA (Strand displacement amplification), HDA (helicase dependent amplification, with heat-labile or heat-stabile enzymes), RPA (recombinase polymerase amplification), LAMP (Loop-mediated amplification); or SMAP (SMart Amplification Process). These technologies make use of enzymes, proteins, primers and accessory molecules that are well known to those skilled in the art. The polymerases include any of a Thermus thermophilus (Tth) DNA polymerase, Thermus acquaticus (Taq) DNA polymerase, Thermotoga maritima (Tma) DNA polymerase, Thermococcus litoralis (Tli) DNA polymerase, Pyrococcus furiosus (Pfu) DNA polymerase, Pyrococcus woesei (Pwo) DNA polymerase, Pyrococcus kodakaraensis KOD DNA polymerase, Thermus filiformis (Tfi) DNA polymerase, Sulfolobus solfataricus Dpo4 DNA polymerase, Thermus pacificus (Tpac) DNA polymerase, Thermus eggertsonii (Teg) DNA polymerase and Thermus flavus (Tfl) DNA polymerase and the polymerases of phages, e.g., Phi29-phage, Phi29 like phages such as Cp-1, PRD-1, Phi 15, Phi 21, PZE, PZA, Nf, M2Y, B103, SF5, GA-1, Cp-5, Cp-7, PR4, PR5, PR722, or L 17. The polymerases also include polymerases from other organisms such as from E. coli, T4, or T7. Other additional proteins may improve the methods, for example helicases, single-stranded binding proteins, other DNA-binding proteins, and recombinases.

In embodiments, PCR is used to perform amplification of the target nucleic acid sequences when present among the nucleic acids to obtain amplicons. PCR is a method that allows exponential amplification of target DNA sequences within a longer double stranded DNA molecule. PCR entails the use of a pair of primers that are complementary to a defined sequence on each of the two strands of the DNA. These primers are extended by a DNA polymerase so that a copy is made of the designated sequence. After making this copy, the same primers can be used again, not only to make another copy of the input DNA strand but also of the short copy made in the first round of synthesis. This leads to logarithmic amplification.

A primer is an oligonucleotide capable of acting as a point of initiation of DNA synthesis under conditions in which synthesis of a primer extension product complementary to a nucleic acid strand is induced, i.e., either in the presence of four different nucleoside triphosphates and an agent for extension (e.g., a DNA polymerase or reverse transcriptase) in an appropriate buffer and at a suitable temperature. A primer may be single-stranded DNA. The appropriate length of a primer depends on the intended use of the primer but may range from 6 to 50 nucleotides, such as from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template.

A forward primer is a primer that is capable of hybridizing to a region of DNA along the 5′ (coding) strand of DNA. A reverse primer is a primer that is capable of hybridizing to a region of DNA along the 3′ (non-coding) strand of DNA. A primer set or primer pair is a specific combination of at least one forward primer and at least one reverse primer. A primer is specific for a target sequence if, when used in an amplification reaction under sufficiently stringent conditions, the primer hybridizes primarily only to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in most cases.

Hybridization conditions can be chosen under which the primer can form stable duplexes only with a target sequence. Thus, the use of target-specific primers under suitably stringent amplification conditions enables the specific amplification of those target sequences which contain the target primer binding sites. The use of sequence-specific amplification conditions enables the specific amplification of those target sequences which contain the complementary primer binding sites. Complementary refers to instances where a nucleic acid molecule can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides. It is understood in the art that a nucleic acid molecule need not be 100% complementary to a target nucleic acid sequence to be specifically hybridizable. That is, two or more nucleic acid molecules may be less than fully complementary and is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing of 5, 6, 7, 8, 9, or 10 nucleotides between the first and second nucleic acid molecules represents 50%, 60%, 70%, 80%, 90%, and 100% complementarity, respectively. “Perfectly” or “fully” complementary nucleic acid molecules means those in which all the contiguous residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

Hybridization refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which only fully complementary nucleic acid strands will hybridize are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art can determine duplex stability empirically considering a number of variables including, for example, the length and base pair composition of the oligonucleotides, ionic strength, and incidence of mismatched base pairs, following the guidance provided by the art. See, e.g., Sambrook et al., (1989) Molecular Cloning—A Laboratory Manual (Cold Spring Harbor Laboratory, Cold Spring Harbor, N.Y.); and Wetmur (1991) Critical Review in Biochem. and Mol. Biol. 26(3/4):227-259.

An amplicon is the DNA sequence generated by a PCR (e.g., qPCR) reaction. In that respect, an amplicon is a PCR product. A target nucleic acid sequence (or “target, “target sequence”, “target region”, or “target nucleic acid”) refers to a region or subsequence of a nucleic acid which is to be amplified and detected. A reaction mixture is a solution containing reagents necessary to carry out a given reaction. And an amplification reaction mixture, which is a solution containing reagents necessary to carry out an amplification reaction, typically contains oligonucleotide primers and a DNA polymerase or ligase in a suitable buffer. A PCR reaction mixture typically contains oligonucleotide primers, a DNA polymerase (most typically a thermostable DNA polymerase), dNTPs, and a divalent metal cation in a suitable buffer. dNTPs are deoxyribose nucleotide triphosphates. They are the building blocks added during a PCR in order to synthesize a new strand. Examples are deoxyadenosine triphosphate (dATP), deoxythymidine triphosphate (dTTP), deoxycytidine triphosphate (dCTP), and deoxyguanosine triphosphate (dGTP) (and/or deoxyuridine triphosphate (dUTP)).

A reaction mixture is referred to as complete if it contains all reagents necessary to enable the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction components.

Real-time PCR, also called quantitative real time PCR, quantitative PCR (Q-PCR/qPCR), or kinetic polymerase chain reaction, is a type of PCR that is used to amplify and simultaneously quantify a targeted DNA molecule. qPCR enables both detection and quantification (as absolute number of copies or relative amount when normalized to DNA input or additional normalizing genes) of a specific sequence in a DNA sample. The procedure follows the general principle of PCR and its key feature is that the amplified DNA is quantified as it accumulates in the reaction in real time after each amplification cycle.

In qPCR, DNA dyes or probes (e.g., fluorescent probes) can be added to the PCR mixture before amplification and used to analyze PCR products (amplicons) during amplification. Sample analysis may occur concurrently with amplification in the same tube within the same instrument. This combined approach decreases sample handling, saves time, and greatly reduces the risk of product contamination for subsequent reactions (as there is no need to remove the samples from their closed containers for further analysis).

The formation of PCR products is monitored in each cycle of the qPCR. The amplification may be measured in thermocyclers which have additional devices for measuring fluorescence signals during the amplification reaction. Several types of real time detection thermocyclers are known in the art.

An example of an instrument capable of performing multiplex real time PCR is the LightCycler (Roche Diagnostics GmbH, Cat. No. 3 531 414 201). It is a fast PCR system enabling kinetic on-line PCR quantification and subsequent analysis of PCR-product melting curves. The optical system of the LightCycler version 2.0 contains one light source, a blue light emitting diode (470 nm LED) and six detection channels. A defined signal threshold is determined for all reactions to be analyzed and the number of cycles Cp required to reach this threshold value is determined for the target nucleic acid as well as for the reference nucleic acids such as the standard or housekeeping gene. The absolute or relative copy numbers of the target molecule can be determined on the basis of the Cp values obtained for the target nucleic acid and the reference nucleic acid. The fluorescence emitted by a sample is separated by a set of dichroic mirrors and filters into different wavelengths that can be recorded in one of the six detection channels. Due to the fluorescent compounds (which are commercially available), this allows detection of the double-stranded DNA-binding dye SYBR Green I, dual color detection with the TAQMAN probe format and 4-color detection with the hybridization probe (Hybprobe) format. Details of the LIGHTCYCLER system are disclosed in WO 97/46707, WO 97/46712 and WO 97/46714.

Similar to the LightCycler system, the Corbett Rotor-Gene Real time PCR Thermocycler (www.corbettresearch.com) is a 4 channel multiplexing system comprising 4 different LEDs as excitation sources and corresponding photodiodes as fluorescent detection units. Another real time PCR instrument is the Biorad iQ Multi-color Real time PCR detection system (Cat. No: 170-8740), which allows for a fluorophore excitation and emission from 400 nm to 700 nm. The system is based on a conventional multiwell heating block for thermocycling, a tungsten lamp as an excitation source, a filter wheel for providing appropriate excitation wavelengths, a second filter wheel for selecting appropriate emission wavelengths and a CCD camera as a detection unit. The instrument has successfully been used in a multiplex assay for the detection of 4 different amplicons generated from targets with more or less equimolar concentrations, using four differently labeled TaqMan probes in the same reaction vessel (Pedersen, S., Bioradiations 107 (2001) 10-11).

In general, there exist different formats for real time detection of amplified DNA, of which the following are well known and commonly used in the art.

DNA Binding Dye Formats: Since the amount of double-stranded amplification product usually exceeds the amount of nucleic acid originally present in the sample to be analyzed, double-stranded DNA specific dyes may be used, which upon excitation with an appropriate wavelength show enhanced fluorescence only if they are bound to double-stranded DNA. Preferably, only those dyes may be used which, like SybrGreenI, for example, do not affect the efficiency of the PCR reaction. Other formats known in the art require the design of a fluorescent labeled hybridization probe (which may only emit fluorescence upon binding to its target nucleic acid).

TaqMan Probe: A single-stranded hybridization probe is labeled with two components. When the first component is excited with light of a suitable wavelength, the absorbed energy is transferred to the second component, the so-called quencher, according to the principle of fluorescence resonance energy transfer. During the annealing step of the PCR reaction, the hybridization probe binds to the target DNA and is degraded by the 5′-3′ exonuclease activity of the Taq Polymerase during the subsequent elongation phase. As a result, the excited fluorescent component and the quencher are spatially separated from one another and thus a fluorescence emission of the first component can be measured. See, e.g., U.S. Pat. No. 5,538,848.

Molecular Beacons: These hybridization probes are also labeled with a first fluorescent component and with a quencher such that the labels may be located at both ends of the probe. As a result of the secondary structure of the probe, both components are in spatial vicinity in solution. After hybridization to the target nucleic acids, both components are separated from one another such that the fluorescence emission of the first component can be measured after excitation with light of a suitable wavelength. See, e.g., U.S. Pat. No. 5,118,801.

Single LabelProbe (SLP) Format: This detection format includes a single oligonucleotide labeled with a single fluorescent dye at either the 5′- or 3′-end (WO 02/14555). Two different designs can be used for oligo labeling: G-Quenching Probes and Nitroindole-Dequenching probes. In the G-Quenching embodiment, the fluorescent dye is attached to a C at the 5′- or 3′-end of the oligonucleotide. Fluorescence decreases significantly when the probe is hybridized to the target, in case two G's are located on the target strand opposite to C and in position 1 aside of complementary oligonucleotide probe. In the Nitroindole-Dequenching embodiment, the fluorescent dye is attached to Nitroindole at the 5′- or 3′-end of the oligonucleotide. Nitroindole decreases the fluorescent signaling of the free probe. Fluorescence increases when the probe is hybridized to the target DNA due to a dequenching effect.

FRET Hybridization Probes: The FRET Hybridization Probe test format is known to be useful for all kinds of homogenous hybridization assays (Matthews, J. A., and Kricka, L. J., Analytical Biochemistry 169 (1988) 1-25. It is characterized by a pair of two single-stranded hybridization probes which are used simultaneously and are complementary to adjacent sites of the same strand of the amplified target nucleic acid. Both probes are labeled with different fluorescent components. When excited with light of a suitable wavelength, a first component transfers the absorbed energy to the second component according to the principle of fluorescence resonance energy transfer such that a fluorescence emission of the second component can be measured when both hybridization probes bind to adjacent positions of the target molecule to be detected.

When annealed to the target sequence, the hybridization probes must sit very close to each other in a head to tail arrangement. The gap between the labeled 3′ end of the first probe and the labeled 5′ end or the second probe is typically as small as possible, i.e. 1-5 bases. This allows for a close vicinity of the FRET donor compound and the FRET acceptor compound, which is typically 10-100 Angstroms.

As an alternative to monitoring the increase in fluorescence of the FRET acceptor component, it is also possible to monitor fluorescence decrease of the FRET donor component as a quantitative measurement of hybridization events.

In embodiments, the FRET Hybridization Probe format may be used in real time PCR in order to detect the amplified target DNA. Besides PCR and real time PCR, FRET hybridization probes are used for melting curve analysis. In such an assay, the target nucleic acid is amplified first in a typical PCR reaction with suitable amplification primers. The hybridization probes may already be present during the amplification reaction or added subsequently. After completion of the PCR reaction, the temperature of the sample is constitutively increased, and fluorescence is detected as long as the hybridization probe was bound to the target DNA. At melting temperature, the hybridization probes are released from their target, and the fluorescent signal decreases to the background level. This decrease is monitored with an appropriate fluorescence versus temperature-time plot such that a first derivative value can be determined at which the maximum of fluorescence decrease is observed.

All probe based detection formats can be “multiplexed”. More precisely, in one reaction vessel, multiple targets may become amplified with multiple pairs of amplification primers and detected with multiple hybridization probes. In embodiments, the multiple probes are labeled with different detectable fluorescent components in order to detect and discriminate the multiple targets which may be found in the sample. For multiplex detection with the FRET hybridization probe format, it is possible that fluorescein or fluorescein derivatives are used as a FRET donor moiety in combination with different FRET acceptor moieties such as Cy-5, LC-Red-640, or LC-Red 705.

### Target Nucleic Acid Sequences

In embodiments, the target nucleic acid sequences include a Psilocybe cubensis target nucleic acid sequence (e.g., ITS, PsiK, PsiM, PsiH, and/or PsiD) and a target nucleic acid sequence from at least one other mushroom species subject to misidentification as Psilocybe cubensis (e.g., an amatoxin target nucleic acid sequence from Galerina marginata and/or a target nucleic acid sequence, such as an ITS sequence, from a Psilocybe species linked to wood lovers paralysis (WLP)). In embodiments, the target nucleic acid sequences include any sequence of a (i) bacterial (e.g., 16S, E. coli, and/or Salmonella) nucleic acid, (ii) Psilocybe (e.g., PsiK, PsiM, PsiH, and/or PsiD) nucleic acid, and/or (iii) fungal (e.g., Internal Transcribed Spacer) nucleic acid. Exemplary target nucleic acids are described in more detail below.

Of note, sequence identities are discussed herein with respect to nucleic acid sequences (e.g., target nucleic acid sequences, primers, probes, etc.). Sequence identities can be calculated using various publicly available software tools developed by NCBI (Bethesda, Maryland, USA) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the nucleic acid sequences are also contemplated herein.

### Amatoxin Target Sequences

Exemplary amatoxin target nucleic acid sequences (from Galerina marginata) have the following sequences:

(SEQ ID NO: 1)
GCAACGACATTTGCTGAACGTGTCCCGCCTGTCGTTGGCCCG
CCGCTTTAACACGAAGTGGGCAACGATACTTATCGACGATGT
TAGGCTTGG
(SEQ ID NO: 2)
CTCCAATCCTCCAACCATCAACTCAACCAAAGATCTTCGCCC
TTGCCTTAATATCTGCCATGTTCGACACCAACTCCACTCGTC
TTCCAAT

In embodiments, other amatoxin target nucleic acid sequences are at least 85%, 86%, 87%, 88%, 89%, 90%, 91%, 92%, 93%, 94%, 95%, 96%, 97%, 98%, or 99% identical to one of these target nucleic acid sequences.

### Wood Lovers Paralysis (WLP) Target Sequences

An exemplary wood lovers paralaysis target sequence is an Internal Transcribed Spacer (ITS) target nucleic acid sequence from a Psilocybe species linked to wood lovers paralysis (WLP), which has the following sequence:

(SEQ ID NO: 3)
AGACGTTGAAACTGGATAGGAGAGGGACTTGTCCTTCAAGTTGAAGGWT
TTTTCGGCGCTCTACGTTTTCATATACCCCAAAGAATGTAACAGAATGT
ATCTTAWGGCTTTATGCCTATAAACTATATACAACTTTCA

In embodiments, other wood lovers paralysis (WLP) target nucleic acid sequences are at least 85%, 86%, 87%, 88%, 89%, 90%, 91%, 92%, 93%, 94%, 95%, 96%, 97%, 98%, or 99% identical to this sequence.

### Bacterial Target Sequences

It is known that certain gene sequence fragments within most bacteria are essentially identical. These sequence fragments are called “conserved” regions. Because of these conserved regions, it is possible to use consensus primers, which bind to these regions, to amplify nucleic acids of bacteria as a class of microorganisms. The conserved bacterial regions include the 16S region, 23S region, and 5S region.

An exemplary conserved 16S target nucleic acid sequence has the following sequence:

(SEQ ID NO: 4)
TCCTACGGGAGGCAGCAGTGGGGAATATTGCACAATGGGCGCAAGCCTG
ATGCAGCCATGCCGCGTGTATGAAGAAGGCCTTCGGGTTGTAAAGTACT
TTCAGCGGGGAGGAAGGGAGTAAAGTTAATACCTTTGCTCATTGACGTT
ACCCGCAGAAGAAGCACCGGCTAACTCCGTGCCAGCAGCCGCGGTAATA
CGGAGGGTGCAAGCGTTAATCGGAATTACTGGGCGTAAAGCGCACGCAG
GCGGTTTGTTAAGTCAGATGTGAAATCCCCGGGCTCAACCTGGGAACTG
CATCTGATACTGGCAAGCTTGAGTCTCGTAGAGGGGGGTAGAATTCCAG
GTGTAGCGGTGAAATGCGTAGAGATCTGGAGGAATACCGGTGGCGAAGG
CGGCCCCCTGGACGAAGACTGACCTCAAGGGTGCCAAAGCGTTGGGGAG
CAAACAGGATTAGATACCCTGGTAGTCC

In embodiments, other conserved 16S target nucleic acid sequences are at least 85%, 86%, 87%, 88%, 89%, 90%, 91%, 92%, 93%, 94%, 95%, 96%, 97%, 98%, or 99% identical to this sequence.

More specific bacterial target nucleic acid sequences may be amplified and detected. For instance, the bacterial target nucleic acid sequence may be an Escherichia coli or Salmonella target nucleic acid sequence.

Detection of the microorganism E. coli has been considered as an indicator of the possible presence of enteric pathogens. Indeed, certain E. coli strains are pathogenic themselves.

An exemplary E. coli target nucleic acid sequence has the following sequence:

(SEQ ID NO: 5)
CAGCGTGGTGGCAAAAACGGATACCGGCAAACATAATGCAATCAAGGTG
GTCACGATGC

In embodiments, other E. coli target nucleic acid sequences are at least 85%, 86%, 87%, 88%, 89%, 90%, 91%, 92%, 93%, 94%, 95%, 96%, 97%, 98%, or 99% identical to this sequence.

Salmonella is a genus of bacteria that are causative of severe infections, notably of food or beverage toxi-infections, leading to bacterial enteric illness in both humans and animals, more particularly to salmonellosis, which include gastroenteritis, as well as typhoid and paratyphoid fevers.

An exemplary Salmonella target nucleic acid sequence has the following sequence:

(SEQ ID NO: 6)
TGGTTTCGATTCGGAAGCGGGTTATCGCCGTCAGGAGGCATTACGAAAA
GAAAATAACATTGGAACAAAAATGGGGAACTTCTCATTCTTCAGCGAAG
AGATGACTGACCCGCTGGTCGCGTTCGCCGGGCAGTGGCGACCAGATCT
CATCG

In embodiments, other Salmonella target nucleic acid sequences are at least 85%, 86%, 87%, 88%, 89%, 90%, 91%, 92%, 93%, 94%, 95%, 96%, 97%, 98%, or 99% identical to this sequence.

### Psilocybe Target Sequences

In embodiments, Psilocybe cubensis target nucleic acid sequences include an Internal Transcribed Spacer (ITS) target nucleic acid sequence. In that respect, an exemplary Psilocybe cubensis ITS target nucleic acid sequence has the following sequence:

(SEQ ID NO: 7)
AGATCATTGTTTTTGGAAGCTGGATTGAAGTCAGAGATTACTCTCTGAT
GAATTGAAGGCTTTCTCAATGATGGTCTACGTTTTCATATACTCCAATG
AATGTAACAGAATGTATCTATATGGCCTTGTGCCTATAAAACAATATAC
AA

In embodiments, other Psilocybe cubensis ITS target nucleic acid sequences are at least 85%, 86%, 87%, 88%, 89%, 90%, 91%, 92%, 93%, 94%, 95%, 96%, 97%, 98%, or 99% identical to this sequence.

In embodiments, Psilocybe target nucleic acids (e.g., P. cubensis target nucleic acids) include a sequence from any of a PsiK, PsiM, PsiH, or PsiD nucleic acid. In embodiments, the target nucleic acid sequences include sequences from any combination of PsiK, PsiM, PsiH, or PsiD nucleic acids. For instance, the Psilocybe target nucleic acid sequence may include a target nucleic acid sequence from each of PsiK and PsiM or may include a target nucleic acid sequence from each of PsiK, PsiM, and PsiD. Further, the target nucleic acid sequence may include a target nucleic acid sequence from each of PsiK, PsiM, PsiH, and PsiD.

PsiK A PsiK nucleic acid (gene) encodes a Psilocybe PsiK enzyme, which is a kinase (e.g., 4-hydroxytryptamine kinase). The PsiK kinase can catalyze the phosphorylation of the phenolic oxygen of 4-hydroxytryptamine to norbaeocystin. The PsiK kinase can also catalyze the phosphorylation of psilocin to psilocybin.

An exemplary PsiK target nucleic acid sequence has the following sequence:

(SEQ ID NO: 8)
AGTGGGGGAGCGAGGAAGAAAGGATAAATTTTGTGAAAAAGGGGGTAGC
TGCCTTTCACGACGCCAGGGGCAACAACGACAATGGGGAAATTACGTCT
ACCTTACTGAAGGAATCATCCACTGCG

In embodiments, other PsiK target nucleic acid sequences are at least 85%, 86%, 87%, 88%, 89%, 90%, 91%, 92%, 93%, 94%, 95%, 96%, 97%, 98%, or 99% identical to this sequence.

PsiM: A PsiM nucleic acid (gene) encodes a Psilocybe PsiM enzyme, which is a methyl transferase (e.g., psilocybin synthase). The PsiM methyl transferase can catalyze the alkylation of the primary amine in norbaeocystin to baecystin. The PsiM methyl transferase can catalyze another alkylation when the secondary amine of baecystin becomes a tertiary amine of psilocybin.

An exemplary PsiM target nucleic acid sequence has the following sequence:

(SEQ ID NO: 9)
GCACTGGCGTGGTGTTGTCTTCACTACTTTGGACTTGGGTTGCTCCTAC
GTGCACAAAGTGAGAGTAAATTTCTCGTCAGGCATTATCTCAAGAACTA
TCATTACGATTACGCTCAGCAAGATGGGGGTCAAGTCGCCGTTATCGAA
GCATTTTCCA

In embodiments, other PsiM target nucleic acid sequences are at least 85%, 86%, 87%, 88%, 89%, 90%, 91%, 92%, 93%, 94%, 95%, 96%, 97%, 98%, or 99% identical to this sequence.

PsiH: A PsiH nucleic acid (gene) encodes a Psilocybe PsiH enzyme, which is a monooxygenase (e.g., tryptamine 4-monooxygenase). The PsiH monooxygenase can catalyze the oxidative hydroxylation of the phenyl ring of tryptamine to 4-hydroxytryptamine.

PsiD: A PsiD nucleic acid (gene) encodes a Psilocybe PsiD enzyme, which is a decarboxylase (e.g., L-tryptophan decarboxylase). The PsiD decarboxylase can catalyze the decarboxylation of an aliphatic carboxylic acid (i.e., release carbon dioxide) so as to catalyze L-tryptophan to tryptamine and 4-hydroxy-L-tryptophan to 4-hydroxytryptamine.

An exemplary PsiD target nucleic acid sequence has the following sequence:

(SEQ ID NO: 10)
ACCCGTCAATGGGACAATCGTCAAAATCATCAACGTTCCAGGTACCTAC
TTTGCGCAAGCCCCGAGCACGATTGGCGACCCTATCCCGGATAACGATT
ACGACCCACCTCCTTACCTTAAGTCTCTTGTCTACTTCTCTAATATTGC
CGCA

In embodiments, other PsiD target nucleic acid sequences are at least 85%, 86%, 87%, 88%, 89%, 90%, 91%, 92%, 93%, 94%, 95%, 96%, 97%, 98%, or 99% identical to this sequence.

### Fungal Target Sequences

It is known that certain gene sequence fragments within most fungus (e.g., yeast and mold) are essentially identical. These sequence fragments are termed “conserved” regions. Because of these conserved regions, it is possible to use consensus primers, which bind to these regions, to amplify nucleic acids of fungi as a class of microorganisms (e.g., total yeast and mold).

The fungal target nucleic acid sequences include sequences of the mitochondrial small subunit ribosomal RNA genes (SSU rDNA) or the Internal Transcribed Spacer (ITS) sequences of the nuclear ribosomal RNA gene regions. Fungal rRNA genes are organized in units, each of which encodes three mature subunits of 18S (small subunit), 5.8S, and 28S (large subunit). These subunits are separated by two Internal Transcribed Spacers, ITS1 and ITS2. In addition, the transcriptional units are separated by non-transcribed spacer sequences (NTSs).

An exemplary fungal ITS target nucleic acid sequence has the following sequence:

(SEQ ID NO: 11)
GCATCGATGAAGAACGCAGCGAAATGCGATAACTAATGTGAATTGCAGA
ATTCAGTGAATCATCGAGTCTTTGAACGCACATTGCGCCCCCTGGTATT
CCGGGGGGCATGCCTGTCCGAGCGTCATTGCTGCCCTCAAGCCCGGCTT
GTGTGTTGGGTCGCCGTCCCCCTCTCCGGGGGGACGGGCCCGAAAGGCA
GCGGCGGCACCGCGTCCGATCCTCGAGCGTATGGGGCTTTGTCACATGC
TCTGTAGGATTGGCCGGCGCCTGCCGACGTTTTCCAACCATTCTTTCCA
GGTTGACCTCGGATCAGGTAGGGATACCCGCTGAACTTAAGCATATCAA
TAAGCGGAGGAAAAGAAACCAACCGGGATTGCCTCAGTAACGGCGAGTG
AAGCGGCAAGAGCTCAAAT

In embodiments, other fungal ITS target nucleic acid sequences are at least 85%, 86%, 87%, 88%, 89%, 90%, 91%, 92%, 93%, 94%, 95%, 96%, 97%, 98%, or 99% identical to this sequence.

### Exemplary Primers and Probes

In embodiments, primers for amplifying one or more target nucleic acid sequences (i.e., primers specific for the target nucleic acid sequences) include primers that hybridize with primer-binding sequences found within a Psilocybe cubensis target nucleic acid sequence (e.g., ITS, PsiK, PsiM, PsiH, and/or PsiD) and a target nucleic acid sequence from at least one other mushroom species subject to misidentification as Psilocybe cubensis (e.g., an amatoxin target nucleic acid sequence from Galerina marginata and/or a target nucleic acid sequence, such as an ITS sequence, from a Psilocybe species linked to wood lovers paralysis (WLP)). In embodiments, primers for amplifying one or more target nucleic acid sequences (i.e., primers specific for the target nucleic acid sequences) include primers that hybridize with primer-binding sequences found within any of a (i) bacterial (e.g., 16S, E. coli, and/or Salmonella) target nucleic acid sequence, (ii) Psilocybe (e.g., PsiK, PsiM, PsiH, and/or PsiD) target nucleic acid sequence, and/or (iii) fungal (e.g., Internal Transcribed Spacer) target nucleic acid sequence.

A primer refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least 6, at least 8, at least 10, at least 15, at least 20, at least 25 nucleotides (e.g., 10 to 60 nucleotides) of a target nucleic acid (i.e., will hybridize to a sequence of the target nucleic acid). In general, a primer sequence is identified as being either “complementary” (i.e., complementary to the coding or sense strand (+)), or “reverse complementary” (i.e., complementary to the anti-sense strand (−)). In embodiments, the primer is an oligonucleotide that acts as a point of initiation of a template-directed synthesis using methods such as PCR under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension.” For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

In embodiments, probes for detecting one or more amplicons obtained from amplifying target nucleic acid sequences (i.e., probes specific for the target nucleic acid sequences) include probes that hybridize with probe-binding sequences found within a Psilocybe cubensis target nucleic acid sequence (e.g., ITS, PsiK, PsiM, PsiH, and/or PsiD) and a target nucleic acid sequence from at least one other mushroom species subject to misidentification as Psilocybe cubensis (e.g., an amatoxin target nucleic acid sequence from Galerina marginata and/or a target nucleic acid sequence, such as an ITS sequence, from a Psilocybe species linked to wood lovers paralysis (WLP)). In embodiments, probes for detecting one or more amplicons obtained from amplifying target nucleic acid sequences (i.e., probes specific for the target nucleic acid sequences) include probes that hybridize with probe-binding sequences within amplicons obtained from amplifying a target nucleic acid sequence found within a (i) bacterial (e.g., 16S, E. coli, and/or Salmonella) target nucleic acid sequence, (ii) Psilocybe (e.g., PsiK, PsiM, PsiH, and/or PsiD) target nucleic acid sequence, and/or (iii) fungal (e.g., Internal Transcribed Spacer) target nucleic acid sequence.

The probe may be labeled with a first fluorescent component/moiety/label. This hybridization probe may be any kind of hybridization probe which is used in real time PCR such as TAQMAN probe, a molecular beacon, or a single labeled probe. In other embodiments, the probe comprises at least a second hybridization probe labeled with a second fluorescent component/moiety/label.

Examples of particular fluorophores that can be used in the probes are known to those of skill in the art (see, e.g., U.S. Pat. No. 5,866,366 to Nazarenko et al.). Exemplary fluorophores include 4-acetamido-4′-isothiocyanatostilbene-2,2′disulfonic acid; acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), QFITC (XRITC), -6-carboxy-fluorescein (HEX), and TET (Tetramethyl fluorescein); fluorescamine; IR144; IR1446; Malachite Green isothiocyanate; 4-methylumbelliferone; ortho cresolphthalein; nitrotyrosine; pararosanilin; Phenol Red; B-phycoerythrin; o-phthaldialdehyde; pyrene and derivatives such as pyrene, pyrene butyrate and succinimidyl 1-pyrene butyrate; Reactive Red 4 (CIBACRON™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, N,N,N′,N′-tetramethyl-6-carboxyrhodamine (TAMRA), tetramethyl rhodamine, and tetramethyl rhodamine isothiocyanate (TRITC); sulforhodamine B; sulforhodamine 101 and sulfonyl chloride derivative of sulforhodamine 101 (Texas Red); riboflavin; rosolic acid and terbium chelate derivatives; LightCycler Red 640; Cy5.5; and Cy56-carboxyfluorescein; boron dipyrromethene difluoride (BODIPY); acridine; stilbene; 6-carboxy-X-rhodamine (ROX); Texas Red; Cy3; Cy5, VIC® (Applied Biosystems); LC Red 640; LC Red 705; and Yakima yellow among others.

Other suitable fluorophores include, for example, those available from Molecular Probes (Eugene, Oregon, USA). In embodiments, a fluorophore is used as a donor fluorophore and/or as an acceptor fluorophore.

Acceptor fluorophores are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher) than the maximum absorbance wavelength of the donor fluorophore, and have a fluorescence emission maximum at a wavelength ranging from about 400 to 900 nm. Acceptor fluorophores have an excitation spectrum which overlaps with the emission of the donor fluorophore, such that energy emitted by the donor can excite the acceptor. An acceptor fluorophore may be attached to a nucleic acid molecule.

In embodiments, an acceptor fluorophore is a quencher, such as Dabcyl, QSY7 (Molecular Probes), QSY33 (Molecular Probes), BLACK HOLE QUENCHERS™ (Glen Research), ECLIPSE™ Dark Quencher (Epoch Biosciences), or IOWA BLACK™ (Integrated DNA Technologies). A quencher can reduce or quench the emission of a donor fluorophore. In such an example, instead of detecting an increase in emission signal from the acceptor fluorophore when in sufficient proximity to the donor fluorophore (or detecting a decrease in emission signal from the acceptor fluorophore when a significant distance from the donor fluorophore), an increase in the emission signal from the donor fluorophore can be detected when the quencher is a significant distance from the donor fluorophore (or a decrease in emission signal from the donor fluorophore when in sufficient proximity to the quencher acceptor fluorophore).

Donor Fluorophores (sometimes simply referred to as a “fluorophore”) are fluorophores or luminescent molecules capable of transferring energy to an acceptor fluorophore, thereby generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength, for example greater than about 10³ M⁻¹ cm⁻¹. A donor fluorophore may be attached to a nucleic acid molecule.

Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

In embodiments, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally at least 20 nucleotides in length, such as at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, or more contiguous nucleotides complementary to the target nucleic acid molecule, such as 20-60 nucleotides, 20-50 nucleotides, 20-40 nucleotides, or 20-30 nucleotides.

### Amatoxin Primers and Probes

Exemplary pairs of primers for amatoxin target nucleic acid sequences (from Galerina marginata) have the following sequences:

Amatoxin Forward
(SEQ ID NO: 12)
CTCCAATCCTCCAACCATCAA
Amatoxin Reverse
(SEQ ID NO: 13)
ATTGGAAGACGAGTGGAGTTG
Amatoxin Forward
(SEQ ID NO: 14)
GCAACGACATTTGCTGAACG
Amatoxin Reverse
(SEQ ID NO: 15)
CCAAGCCTAACATCGTCGATAA

In embodiments, a primer has a sequence that is at least 70%, 75%, 80%, 85%, 90%, or 95% identical to one of these primer sequences.

Exemplary probes for an amplicon amplified from an amatoxin target nucleic acid sequence have the following sequences/structures:

Amatoxin Probes
(SEQ ID NO: 16)
/56-ROXN/TCGCCCTTGCCTTAATATCTGCCA/3BHQ_2/
(SEQ ID NO: 17)
/56-ROXN/CGCTTTAACACGAAGTGGGCAACG/3BHQ_2/

In embodiments, a probe has a sequence that is at least 70%, 75%, 80%, 85%, 90%, or 95% identical to one of these probe sequences.

### Wood Lovers Paralysis (WLP) Primers and Probes

An exemplary pair of primers for an ITS target nucleic acid sequence from a Psilocybe species linked to wood lovers paralysis (WLP) have the following sequences:

WLP ITS Forward
(SEQ ID NO: 18)
GTAGACGTTGAAACTGGATAGGAGAG
WLP ITS Reverse
(SEQ ID NO: 19)
AAATTGTATATAGTTTATAGGCATAAAGCCWTA

In embodiments, a primer has a sequence that is at least 70%, 75%, 80%, 85%, 90%, or 95% identical to one of these primer sequences.

An exemplary probe for an amplicon amplified from the ITS target nucleic acid sequence from a Psilocybe species linked to wood lovers paralysis (WLP) has the following sequence/structure:

WLP ITS Probe
(SEQ ID NO: 20)
5Cy5/CTTGTCCTTCAAGTTGAAGGATTTTTCGGCGC/3BHQ_2/

In embodiments, a probe has a sequence that is at least 70%, 75%, 80%, 85%, 90%, or 95% identical to this probe sequence.

### Bacterial Primers and Probes

An exemplary pair of primers for a conserved bacterial 16S target nucleic acid sequence have the following sequences:

16S Forward
(SEQ ID NO: 21)
TACACGACGTTGTAAAACGATCCTACGGGAGGCAGCAGT
16S Reverse
(SEQ ID NO: 22)
AGGATAACAATTTCACACAGGGGACTACCAGGGTATCTAATCCTGTT

In embodiments, a primer has a sequence that is at least 70%, 75%, 80%, 85%, 90%, or 95% identical to one of these primer sequences.

Exemplary probes for an amplicon amplified from a conserved bacterial 16S target nucleic acid sequence have the following sequences/structures:

16S Probes
(SEQ ID NO: 23)
/56-FAM/CGTATTACC/ZEN/GCGGCTGCTGGCAC/3IABkFQ/
(SEQ ID NO: 23)
/56-ROXN/CGTATTACCGCGGCTGCTGGCAC/3IAbRQSp/

In embodiments, a probe has a sequence that is at least 70%, 75%, 80%, 85%, 90%, or 95% identical to one of these probe sequences.

An exemplary pair of primers for an E. coli target nucleic acid sequence have the following sequence:

E. Coli Forward
(SEQ ID NO: 24)
TACACGACGTTGTAAAACGACGCATCGTGACCACCTTGA
E. Coli Reverse
(SEQ ID NO: 25)
AGGATAACAATTTCACACAGGCAGCGTGGTGGCAAAA

In embodiments, a primer has a sequence that is at least 70%, 75%, 80%, 85%, 90%, or 95% identical to one of these primer sequences.

An exemplary probe for an amplicon amplified from the E. coli target nucleic acid sequence has the following sequence/structure:

E. Coli Probe
(SEQ ID NO: 26)
/56-FAM/CCGGCAAAC/ZEN/ATAATGCAATC/3IABKFQ/

In embodiments, a probe has a sequence that is at least 70%, 75%, 80%, 85%, 90%, or 95% identical to this probe sequence.

An exemplary pair of primers for a Salmonella target nucleic acid sequence have the following sequences:

Salmonella Forward
(SEQ ID NO: 27)
TACACGACGTTGTAAAACGACTGGTTTCGATTCGGAAGC
Salmonella Reverse
(SEQ ID NO: 28)
AGGATAACAATTTCACACAGGCGATGAGATCTGGTCGC

In embodiments, a primer has a sequence that is at least 70%, 75%, 80%, 85%, 90%, or 95% identical to one of these primer sequences.

An exemplary probe for an amplicon amplified from the Salmonella target nucleic acid sequence has the following sequence/structure:

Salmonella Probe
(SEQ ID NO: 29)
/5TEX615/CCGTCAGGAGGCATTACGAAAAGA/3IAbRQSp/

In embodiments, a probe has a sequence that is at least 70%, 75%, 80%, 85%, 90%, or 95% identical to this probe sequence.

### Psilocybe Primers and Probes

An exemplary pair of primers for an ITS target nucleic acid sequence from Psilocybe cubensis have the following sequences:

PC ITS Forward
(SEQ ID NO: 30)
TCATTGTTTTTGGAAGCTGGATTGAAGTCA
PC ITS Reverse
(SEQ ID NO: 31)
AAAGTTGTATATTGTTTTATAGGCACAAGGCCAT

In embodiments, a primer has a sequence that is at least 70%, 75%, 80%, 85%, 90%, or 95% identical to one of these primer sequences.

An exemplary probe for an amplicon amplified from the ITS target nucleic acid sequence from Psilocybe cubensis has the following sequence/structure:

PC ITS Probe
(SEQ ID NO: 32)
/5HEX/TACTCTCTGATGAATTGAAGGCTTTCTCAATGATGG/
3BHQ_1/

In embodiments, a probe has a sequence that is at least 70%, 75%, 80%, 85%, 90%, or 95% identical to this probe sequence.

Exemplary pairs of primers for Psilocybe PsiK, PsiM, PsiH, and PsiD target nucleic acid sequences have the following sequences:

PsiK Forward
(SEQ ID NO: 33)
GGAGCGAGGAAGAAAGGATAAA
PsiK Reverse
(SEQ ID NO: 34)
GCAGTGGATGATTCCTTCAGTA
PsiM Forward
(SEQ ID NO: 35)
CGTGGTGTTGTCTTCACTACTT
PsiM Reverse
(SEQ ID NO: 36)
CCCATCTTGCTGAGCGTAAT
PsiD Forward
(SEQ ID NO: 37)
ACCCGTCAATGGGACAATC
PsiD Reverse
(SEQ ID NO: 38)
GAGGTGGGTCGTAATCGTTATC

In embodiments, a primer has a sequence that is at least 70%, 75%, 80%, 85%, 90%, or 95% identical to one of these primer sequences.

Exemplary probes for amplicons amplified from PsiK, PsiM, PsiH, and PsiD target nucleic acid sequences have the following sequences/structures:

PsiK Probes
(SEQ ID NO: 39)
/5HEX/TAGCTGCCT/ZEN/TTCACGACGCCA/3IABKFQ/
(SEQ ID NO: 39)
/5HEX/TAGCTGCCTTTCACGACGCCA/3BHQ_1/
PsiM Probe
(SEQ ID NO: 40)
/5HEX/TGGACTTGG/ZEN/GTTGCTCCTACGTG/3IABKFQ/
(SEQ ID NO: 40)
/5HEX/TGGACTTGGGTTGCTCCTACGTG/3BHQ_1/
PsiD Probe
(SEQ ID NO: 41)
/5HEX/CGAGCACGA/ZEN/TTGGCGACCCTAT/3IABKFQ/

In embodiments, a probe has a sequence that is at least 70%, 75%, 80%, 85%, 90%, or 95% identical to one of these probe sequences.

### Fungal Primers and Probes

An exemplary pair of primers for a fungal ITS region have the following sequences:

Fungal ITS Forward
(SEQ ID NO: 42)
TACACGACGTTGTAAAACGACGCATCGATGAAGAACGCAGC
Fungal ITS Reverse
(SEQ ID NO: 43)
AGGATAACAATTTCACACAGGATTTGAGCTCTTGCCGCTTCA

In embodiments, a primer has a sequence that is at least 70%, 75%, 80%, 85%, 90%, or 95% identical to one of these primer sequences.

An exemplary probe for an amplicon amplified from fungal ITS region has the following sequence/structure:

Fungal ITS Probe
(SEQ ID NO: 44)
/56-FAM/CGCTGAACT/ZEN/TAAGCATATCAATAAGCGG/3IABKFQ/

In embodiments, a probe has a sequence that is at least 70%, 75%, 80%, 85%, 90%, or 95% identical to this probe sequence.

### Exemplary Primer Probe Design Considerations

To design primers and probes, it may be useful to perform sequence alignments of nucleic acid (e.g., gene) regions to identify target nucleic acid sequences based on which regions of the nucleic acid (e.g., gene) of interest are polymorphic (variable) or conserved. Such sequence alignments may be performed using multiple different Psilocybe strains/species/samples (e.g., at least 10, 20, 30, 40, 50, 60, 70, or 80 different Psilocybe strains/species/samples).

In embodiments, a primer or probe is designed so as to hybridize to a region that is conserved such that the region has less than 3%, 2%, 1%, or 0.5% genomic variation in a sequence alignment. In embodiments, any or all target nucleic acid sequences are regions that have less than 3%, 2%, 1%, or 0.5% genomic variation in a sequence alignment.

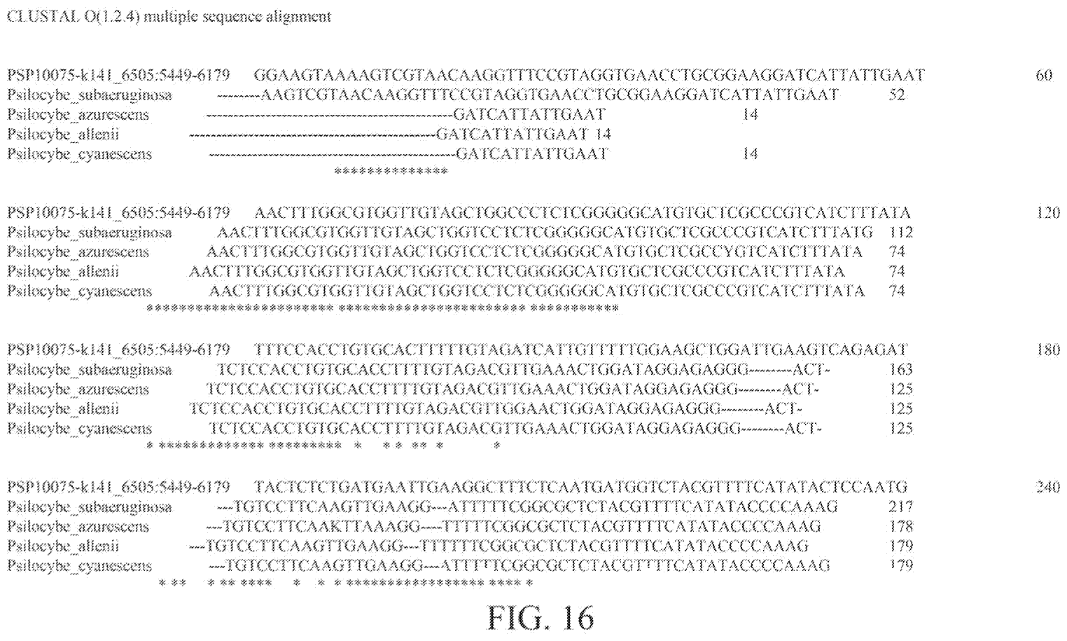

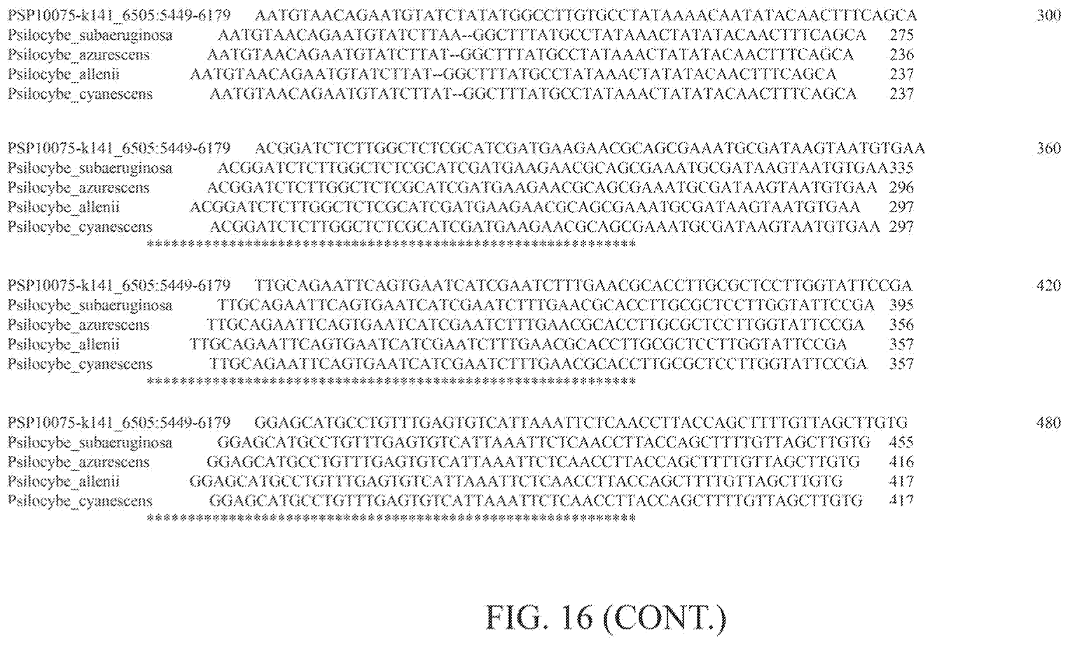

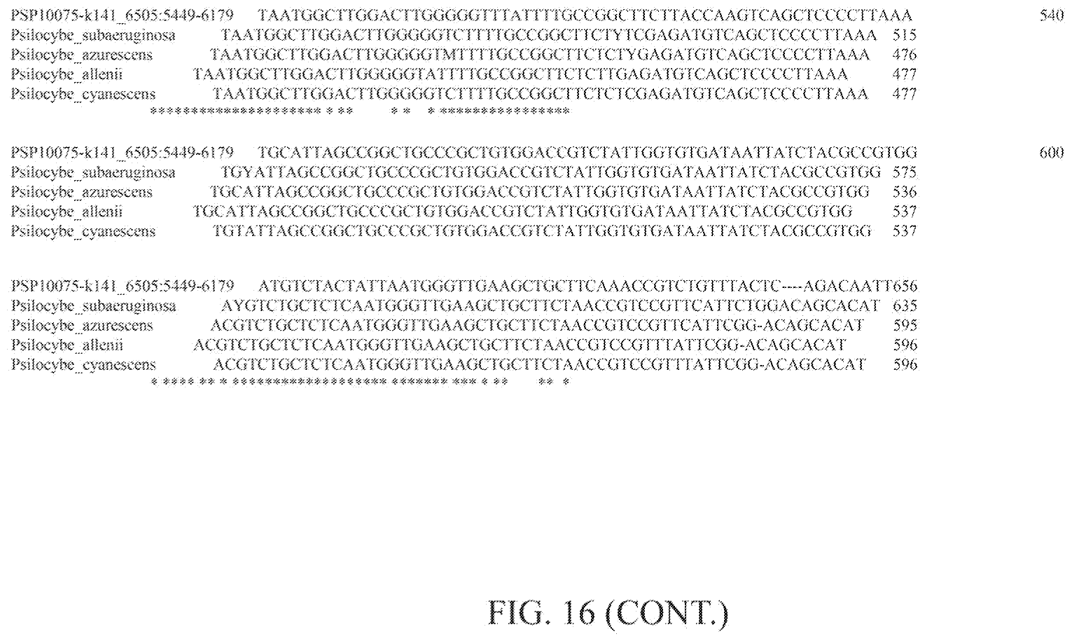

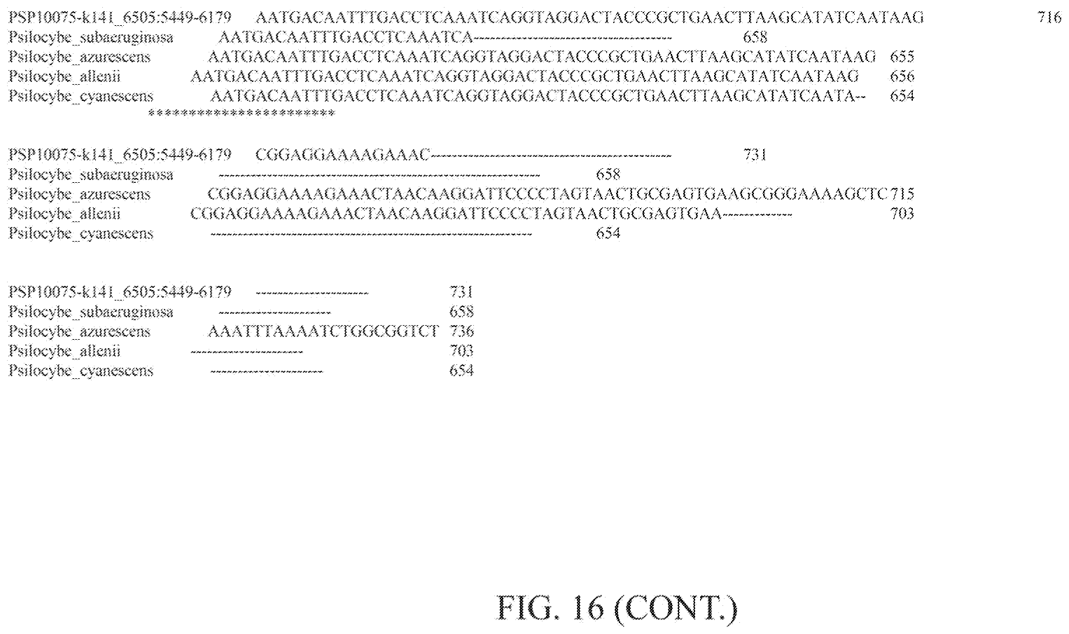

FIG. 16 shows exemplary sequence alignments of Internal Transcribed Spacer (ITS) nucleic acid sequences from Psilocybe species linked to wood lovers paralysis (WLP).

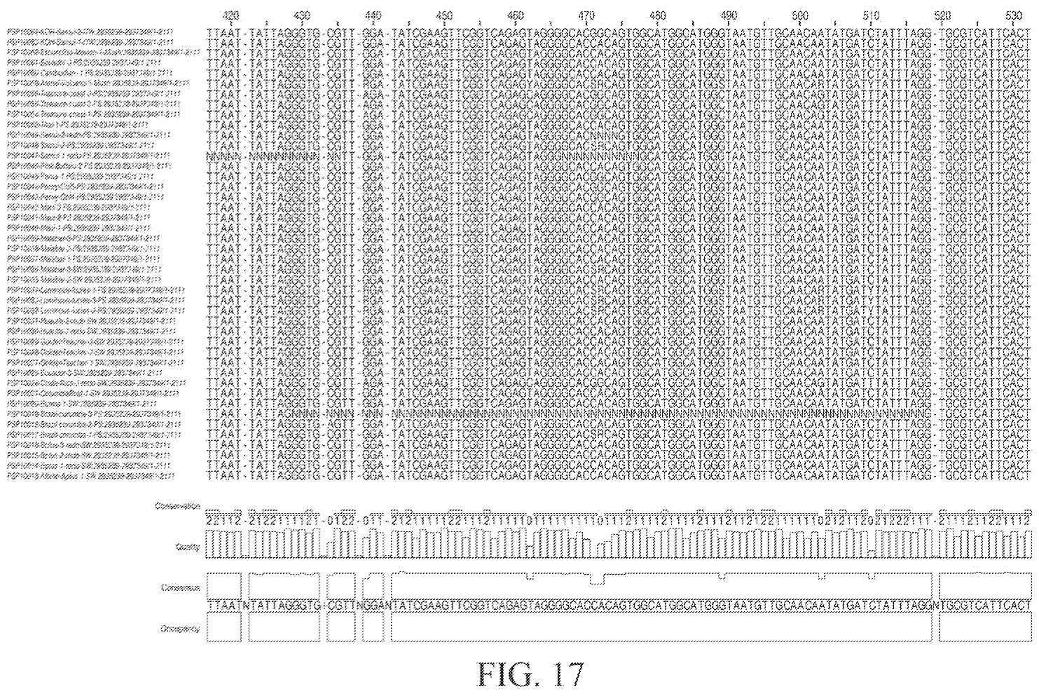

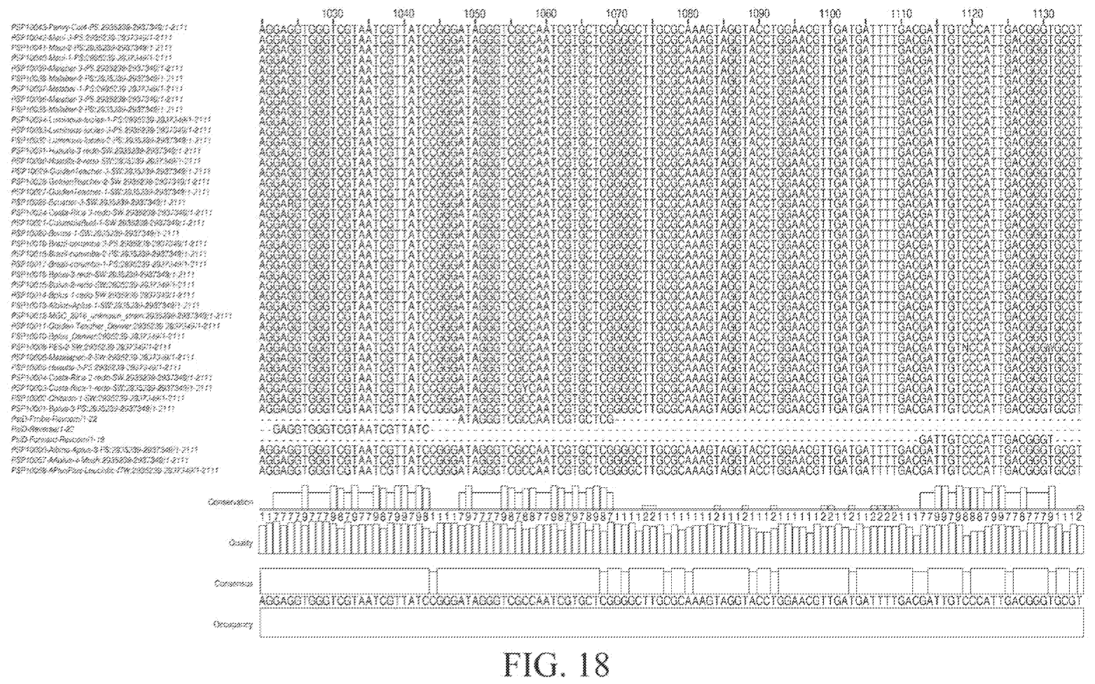

FIG. 17 show exemplary sequence alignments for PsiD regions. In that respect, FIG. 17 shows a sequence alignment indicating polymorphic PsiD regions between different Psilocybe strains/species/samples. And FIG. 18 shows a sequence alignment indicating conserved PsiD regions between different Psilocybe strains/species/samples.

Due to polymorphism, primers and a probe were not designed based on the sequence alignment of FIG. 17. Instead, as shown towards the bottom of FIG. 18's sequence alignment, exemplary primer and probes sequences were designed based on the conserved regions identified in FIG. 18's sequence alignment. Of course, other primer and probes sequences may be designed based on FIG. 18's sequence alignment.

Similar sequence alignments may be helpful for designing primers and probes that hybridize to any of the target nucleic acid sequences of the methods discussed above and the kits discussed below.

### Kits

Kits for performing any of the processes described herein constitute other embodiments. In general, the kits include the primers and/or probes as described herein. The kit may also contain other suitably packaged reagents and other materials needed for the particular assay protocol, for example, controls and polymerizing agents, as well as instructions for conducting the assay/test.

In use, the components of the PCR kit, when applied to the sample, create a reagent mixture which enables the amplification and detection of the target nucleic acid sequences described herein. The reagent mixture thus includes the components of the kit as well as the sample (once provided) which may contain the target nucleic acids of interest.

In embodiments, a kit for assaying (testing) Psilocybe spores, Psilocybe tissue, or a cultured host organism that expresses Psilocybe genes comprises (i) primers for amplifying a bacterial target nucleic acid sequence, a Psilocybe target nucleic acid sequence, and/or a fungal target nucleic acid sequence, and (ii) probes for detecting the amplicons amplified from any of those target nucleic acid sequences.

For instance, the primers may include primers for amplifying a Psilocybe cubensis target nucleic acid sequence and a target nucleic acid sequence from at least one other mushroom species subject to misidentification as Psilocybe cubensis. In embodiments, a kit for discriminating Psilocybe cubensis from at least one other mushroom species subject to misidentification as Psilocybe cubensis comprises (i) primers for amplifying a Psilocybe cubensis target nucleic acid sequence and a target nucleic acid sequence from the other mushroom species and (ii) probes for detecting amplicons of the Psilocybe cubensis target nucleic acid sequence and the target nucleic acid sequence from the other mushroom species. The probes may include a fluorescent label. The kit may further comprise reagents including any of a lysis buffer, magnetic beads, a binding buffer, a wash solution, an elution solution, a DNA polymerase, or dNTPs. The kit may also further comprise instructions for performing an assay (or a test) to discriminate Psilocybe cubensis from the other mushroom species.

In embodiments, the Psilocybe cubensis target nucleic acid sequence may include an Internal Transcribed Spacer (ITS) target nucleic acid sequence and/or any of a PsiK, PsiM, PsiH, or PsiD target nucleic acid sequence. In that respect, the Psilocybe cubensis target nucleic acid sequence may include at least the Internal Transcribed Spacer (ITS) target nucleic acid sequence. The target nucleic acid sequence from the other mushroom species may include any of an amatoxin target nucleic acid sequence or an Internal Transcribed Spacer (ITS) target nucleic acid sequence. The target nucleic acid sequence from the other mushroom species may include both an amatoxin target nucleic acid sequence and an Internal Transcribed Spacer (ITS) target nucleic acid sequence.

In embodiments, the target nucleic acid sequence from the other mushroom species may include an amatoxin target nucleic acid sequence that is, for example, from an amatoxin-producing Galerina species. For instance, the amatoxin target nucleic acid sequence may be from any of Galerina marginata, Galerina venenata, Galerina castaneipes, Galerina fasciculata, Galerina sulcipes, Galerina helvoliceps, Galerina beinrothii, Galerina badipes, Galerina autumnalis, Galerina unicolor, Galerina oregonensis, Galerina physospora, Galerina patagonica, Galerina jaapii, or Galerina triscopa. In embodiments, the target nucleic acid sequence from the other mushroom species may include an Internal Transcribed Spacer (ITS) target nucleic acid sequence from a Psilocybe species linked to wood lovers paralysis (WLP). For instance, the target nucleic acid sequence from the other mushroom species may include an Internal Transcribed Spacer (ITS) target nucleic acid sequence from any of Psilocybe azurescens, Psilocybe cyanescens, Psilocybe allenii, Psilocybe subaeruginosa, Psilocybe stunzii, Psilocybe pelliculosa, or Psilocybe ovoideocystidiata.

In embodiments, a kit for detecting contamination in a Psilocybe sample comprises (i) primers for amplifying a bacterial target nucleic acid sequence, a Psilocybe target nucleic acid sequence, and/or a fungal target nucleic acid sequence, and (ii) probes for detecting the amplicons amplified from any of those target nucleic acid sequences. For instance, the kit may comprise primers for amplifying a bacterial target nucleic acid sequence and a Psilocybe target nucleic acid sequence and probes for detecting amplicons of the bacterial target nucleic acid sequence and the Psilocybe target nucleic acid sequence. The probes may include a fluorescent label. The kit may further comprise reagents including any of a lysis buffer, magnetic beads, a binding buffer, a wash solution, an elution solution, a DNA polymerase, or dNTPs. The kit may also further comprise instructions for performing an assay (or a test) to detect contamination in a Psilocybe sample.

In embodiments, the bacterial target nucleic acid sequence is a 16S target nucleic acid sequence and the Psilocybe target nucleic acid sequence is any of a PsiK, PsiM, PsiH, or PsiD target nucleic acid sequence. That is, the Psilocybe target nucleic acid sequence may be any combination of PsiK, PsiM, PsiH, or PsiD target nucleic acid sequences. For example, the Psilocybe target nucleic acid sequences may include a target nucleic acid sequence from each of PsiK and PsiM or may include a target nucleic acid sequence from each of PsiK, PsiM, and PsiD.

In embodiments, the kit comprises primers for amplifying a fungal target nucleic acid sequence and a probe for detecting amplicons of the fungal target nucleic acid sequence. The fungal target nucleic acid sequence may be an Internal Transcribed Spacer (ITS) target nucleic acid sequence.

In embodiments, the probes have different fluorescent labels based on different amplicons sequences to which the probes hybridize. The probes may have a probe structure that includes a fluorophore and a quencher.

In embodiments, the primers include a primer having a sequence that is at least 70%, 75%, 80%, 85%, 90%, or 95% identical to any one of SEQ ID NOS: 12-15, 18, 19, 21, 22, 24, 25, 27, 28, 30, 31, 33-38, 42, or 43. In embodiments, the probes include a probe having a sequence that is at least 70%, 75%, 80%, 85%, 90%, or 95% identical to any one of SEQ ID NOS: 16, 17, 20, 23, 26, 29, 32, 39-41, or 44.

In embodiments, the target nucleic acid sequences include a target nucleic acid sequence that is at least 85%, 86%, 87%, 88%, 89%, 90%, 91%, 92%, 93%, 94%, 95%, 96%, 97%, 98%, or 99% identical to any one of SEQ ID NOS: 1-3 or 7. In embodiments, the bacterial and Psilocybe target nucleic acid sequences include a target nucleic acid sequence that is at least 85%, 86%, 87%, 88%, 89%, 90%, 91%, 92%, 93%, 94%, 95%, 96%, 97%, 98%, or 99% identical to any one of SEQ ID NOS: 4-6 or 8-10. In embodiments, the fungal target nucleic acid sequence is at least 85%, 86%, 87%, 88%, 89%, 90%, 91%, 92%, 93%, 94%, 95%, 96%, 97%, 98%, or 99% identical to SEQ ID NO: 11.

### Equivalents and Incorporation by Reference

Those skilled in the art will recognize, or be able to ascertain using no more than routine experimentation, many equivalents to the specific embodiments of the invention described herein. Such equivalents are intended to be encompassed by the following claims. All references, including patent documents, disclosed herein are incorporated by reference in their entirety, particularly for the disclosure referenced herein.

### EXAMPLES

Aspects of the present teachings may be further understood in light of the following examples, which should not be construed as limiting in any way. That is, the following examples are provided to illustrate various inventive aspects. They are not intended to be limiting.

### Example 1

Real time PCR (qPCR) for detecting safe-to-consume Psilocybe cubensis: This aPCR approach can discriminate between Psilocybe cubensis and other poisonous mushrooms for which it may be confused. As discussed above, other poisonous mushrooms include Psilocybe species linked to wood lovers paralysis (WLP) and those with an amatoxin gene, such as Galerina marginata. In that respect, sequencing was performed on over 100 Psilocybe genomes (and their ITS sequences were itemized), psilocybin synthesis genes and amatoxin genes from Galerina marginata.

For this exemplary assay, described below are primers that selectively amplify Psilocybe cubensis specific PsiK and PsiM genes and an Internal Transcribed Spacer (ITS) sequence from the Psilocybe cubensis genome. Also, described below are primers that selectively target Psilocybe cubensis and Psilocybe “wood lovers paralysis” ITS regions, respectively.

Primers, probe, and synthetic positive control that can selectively amplify and detect Psilocybe species linked to wood lovers paralysis (WLP) are:

Wood_Lover_ITS_F:
(SEQ ID NO: 18)
GTAGACGTTGAAACTGGATAGGAGAG
Wood_Lovers_ITS_R:
(SEQ ID NO: 19)
AAATTGTATATAGTTTATAGGCATAAAGCCWTA
Wood_Lovers_Probe:
(SEQ ID NO: 20)
5Cy5/CTTGTCCTTCAAGTTGAAGGATTTTTCGGCGC/3BHQ_2/
Wood_Lover_PosCTRL:
(SEQ ID NO: 3)
AGACGTTGAAACTGGATAGGAGAGGGACTTGTCCTTCAAGTTGAAGGWT
TTTTCGGCGCTCTACGTTTTCATATACCCCAAAGAATGTAACAGAATGT
ATCTTAWGGCTTTATGCCTATAAACTATATACAACTTTCA

Primers, probe, and synthetic positive control that can selectively amplify and detect Psilocybe cubensis are:

Cubensis_ITSF:
(SEQ ID NO: 30)
TCATTGTTTTTGGAAGCTGGATTGAAGTCA
Cubensis_ITSR:
(SEQ ID NO: 31)
AAAGTTGTATATTGTTTTATAGGCACAAGGCCAT
Cubensis_ITS_probe:
(SEQ ID NO: 32)
/5HEX/TACTCTCTGATGAATTGAAGGCTTTCTCAATGATGG/3BHQ_1/
P. cubensis_ITS.pctrl:
(SEQ ID NO: 7)
AGATCATTGTTTTTGGAAGCTGGATTGAAGTCAGAGATTACTCTCTGAT
GAATTGAAGGCTTTCTCAATGATGGTCTACGTTTTCATATACTCCAATG
AATGTAACAGAATGTATCTATATGGCCTTGTGCCTATAAAACAATATAC
AA

Primers, probes, and synthetic positive controls that can selectively amplify and detect Galerina amatoxin genes are:

Amatoxin-1-2F:
(SEQ ID NO: 12)
CTCCAATCCTCCAACCATCAA
Amatoxin-1-2R:
(SEQ ID NO: 13)
ATTGGAAGACGAGTGGAGTTG
Amatoxin-1-2P:
(SEQ ID NO: 16)
/56-ROXN/TCGCCCTTGCCTTAATATCTGCCA/3BHQ_2/
Amatoxin-1-1F:
(SEQ ID NO: 14)
GCAACGACATTTGCTGAACG
Amatoxin-1-1R:
(SEQ ID NO: 15)
CCAAGCCTAACATCGTCGATAA
Amatoxin-1-1P:
(SEQ ID NO: 17)
/56-ROXN/CGCTTTAACACGAAGTGGGCAACG/3BHQ_2/
Amal-1 control:
(SEQ ID NO: 1)
GCAACGACATTTGCTGAACGTGTCCCGCCTGTCGTTGGCCCGCCGCTTTA
ACACGAAGTGGGCAACGATACTTATCGACGATGTTAGGCTTGG
Ama1-2 control:
(SEQ ID NO: 2)
CTCCAATCCTCCAACCATCAACTCAACCAAAGATCTTCGCCCTTGCCTTA
ATATCTGCCATGTTCGACACCAACTCCACTCGTCTTCCAAT

While optional for this exemplary assay, primers and probes that can selectively amplify and detect PsiK and PsiM target nucleic acid sequences (Psilocybe cubensis) are:

PsiM_Forward:
(SEQ ID NO: 35)
CGTGGTGTTGTCTTCACTACTT
PsiM_Reverse:
(SEQ ID NO: 36)
CCCATCTTGCTGAGCGTAAT
PsiM_Probe:
(SEQ ID NO: 40)
/5HEX/TGGACTTGGGTTGCTCCTACGTG/3BHQ_1/
PsiK_Forward:
(SEQ ID NO: 33)
GGAGCGAGGAAGAAAGGATAAA
PsiK_Reverse:
(SEQ ID NO: 34)
GCAGTGGATGATTCCTTCAGTA
PsiK_Probe:
(SEQ ID NO: 39)
/5HEX/TAGCTGCCTTTCACGACGCCA/3BHQ_1/

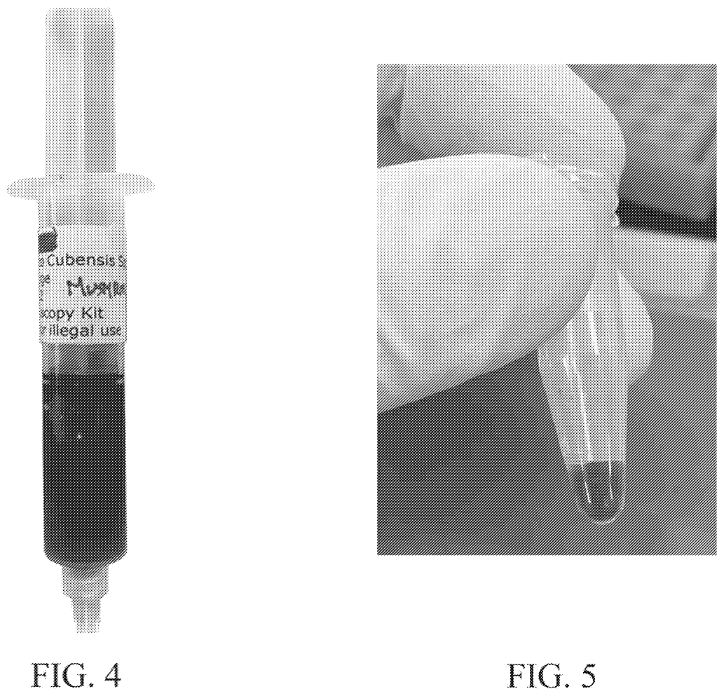

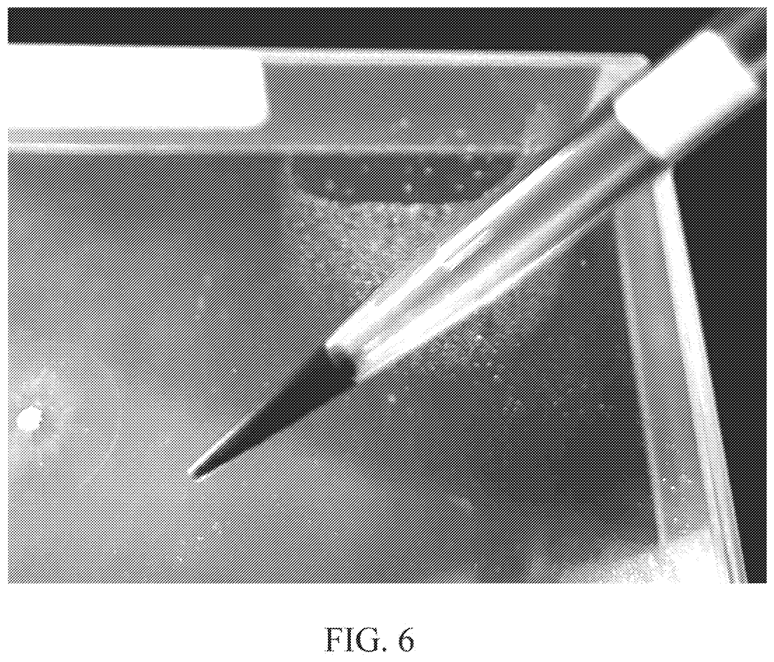

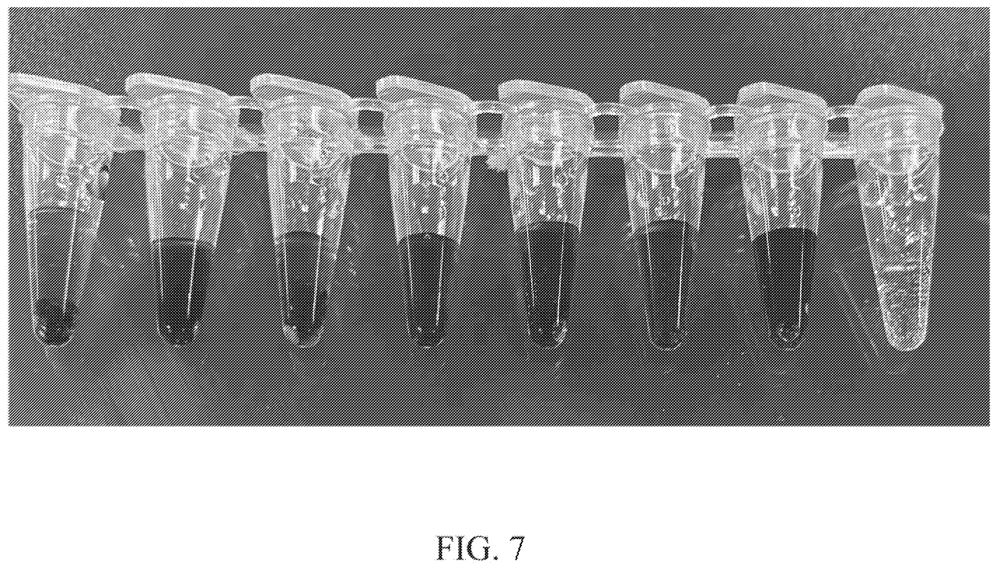

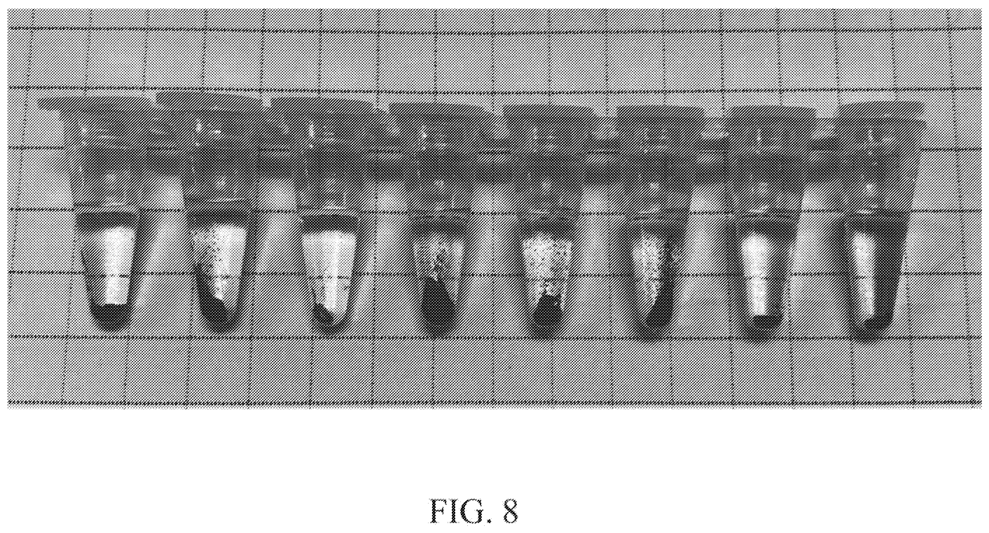

Methods: 1 ml of spores in a spore syringe (Premium Spores, Sporeworks, Inoculate the World, Mushrooms.com) were vortexed as shown in FIG. 4. The spore syringe was spun down into an epitube and 900 ul was removed. FIG. 5 shows the initial 1 ml of spores concentrated into 100 ul with centrifugation. 25 ul of 100 ul pellet is pipetted and placed into 100 ul of Leaf Lysis buffer. As shown in FIG. 6, it can be important to visually confirm the pipette has dark spores since some spores can clump. Each sample is boiled for 6 minutes at 65 C and 2 minutes at 95 C. FIG. 7 shows exemplary boiled spores. Each sample is then spun down and pipetted 5 ul into qPCR reaction. FIG. 8 shows clarity of the centrifuged samples used for qPCR.

qPCR Conditions:
20 reaction master mix
164 ul ddH20
76 ul V3 Mastermix
16 ul Reaction Buffer
2.5 ul Amatoxin Primer/Probe
12.5 ul Wood Lovers ITS Probe
5 ul Cubensis ITS Probe
13.8 ul/reaction
5 ul Boiled DNA/reaction

### Primer and Probe Mixtures:

25 ul 100 uM probe
50 ul 100 uM Forward Primer
50 ul 100 uM Reverse Primer
75 ul ddH20

### Thermal Cycling:

95C 3 minutes
39 Cycles
{
95C 15 seconds
64C for 40 seconds
}

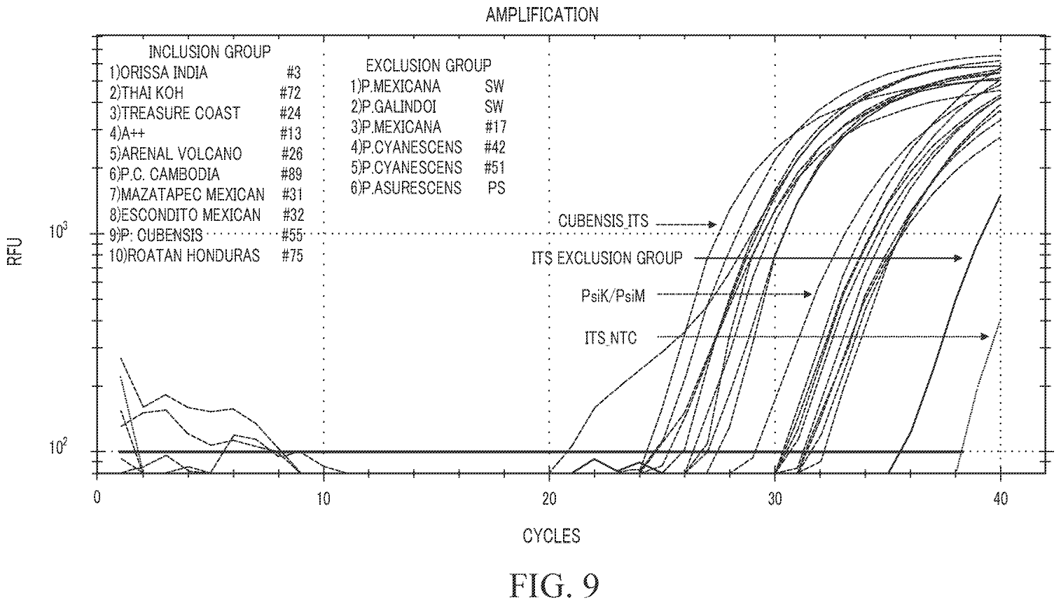

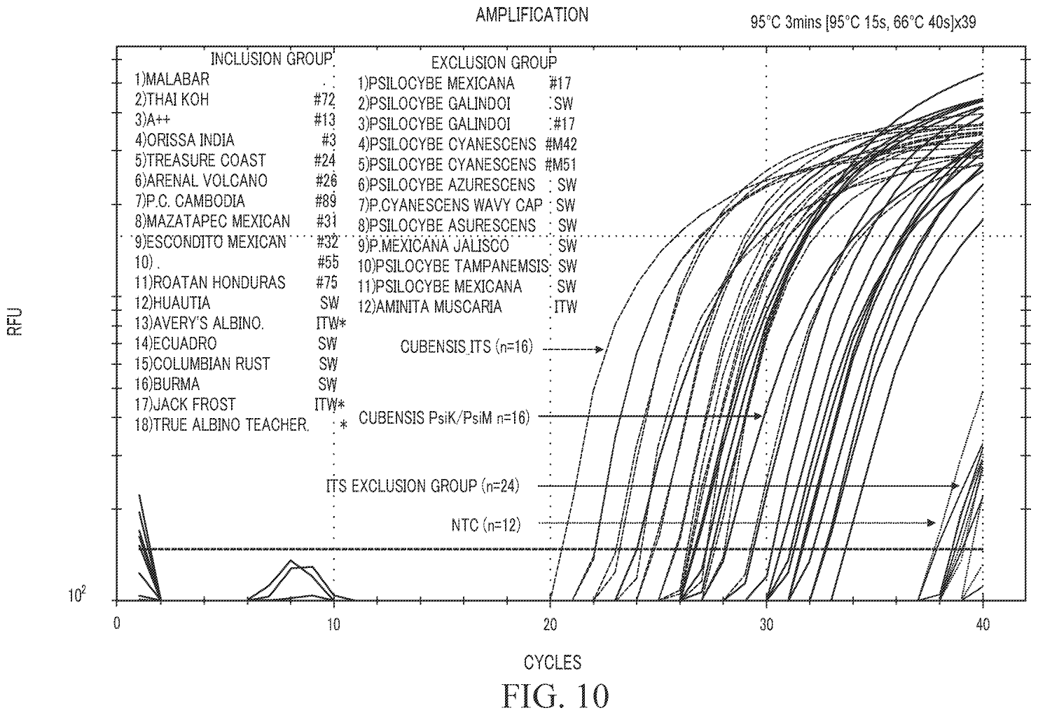

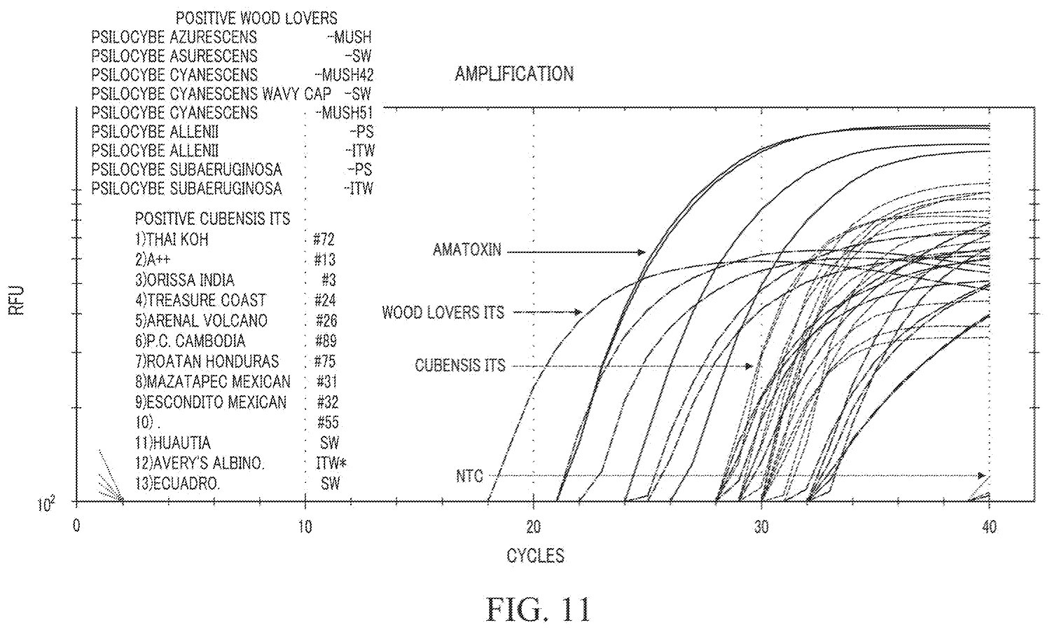

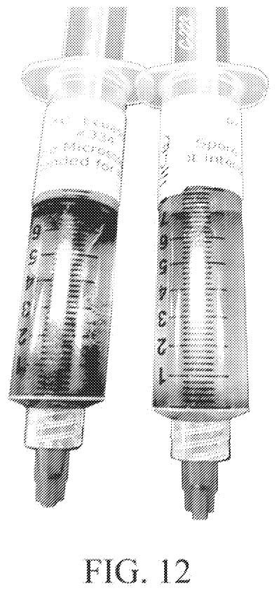

Results: FIG. 9 shows (i) Psilocybe cubensis samples that were amplified with Cubensis ITS qPCR. The Exclusion group did not amplify (except that one late amplification is seen with an Exclusion organism near the No Template Control (NTC) signal). FIG. 10 shows results from an expanded study comparing Cubensis ITS primers with PsiK and PsiM primers. Both primer sets amplified target Psilocybe cubensis and excluded the non-cubensis species. Psilocybe ITS primers have 5CT shift suggesting 2\hat{ }5 or 32 fold higher copy number compared to PsiK and PsiM autosomal genes. The Exclusion organisms failed to amplify significantly above the NTC signal. FIG. 11 shows a triplex qPCR with Amatoxin amplification, Wood Lovers ITS amplification and Cubensis ITS amplification, in which 100% of Exclusion species are properly detected with wood lovers amplicons and not cubensis amplicons.

Conclusion: qPCR assays have been successfully designed that can discriminate (itemize, differentiate, etc.) Psilocybe cubensis from Psilocybe species linked to wood lovers paralysis (WLP) and those with an amatoxin gene, such as Galerina marginata. Thus, the assays can discriminate between Psilocybe cubensis and other poisonous mushrooms for which it may be confused.

### Example 2

DNA isolation from P. cubensis spores: Spores were obtained from 4 vendors (Sporeworks.com, Premiumspores.com, Mushroom.com and InnoculateTheWorld.com). FIG. 12 shows an example of syringes holding an aqueous solution of P. cubensis spores. Spore preparations utilized a modified DNA isolation procedure described in McKernan K. 2021, “Whole genome sequencing of colonies derived from cannabis flowers and the impact of media selection on benchmarking total yeast and mold detection tools,” Zenodo doi:10.5281/zenodo.4759883.

Briefly, 1.4 ml of spores were centrifuged, decanted and resuspended in 200 ul of ddH20. 25 μl of a Thaumatin-like protein was added and incubated at 37° C. (Medicinal Genomics part #420206) for 30 minutes. 12.5 μl of MGC lysis buffer was added and incubated at 65° C. for 30 minutes with 9 steel beads. Vortexing was performed every 7 minutes. Lysed sample were micro-centrifuged and 200 μl of supernatant was aspirated and added to 250 μl of Medicinal Genomics (MGC) binding buffer (MGC part #420001) for magnetic bead isolation. The samples were incubated with the MGC magnetic bead mixture for 10 minutes, magnetically separated and washed two times with 70% ethanol. The beads were dried at 37° C. for 5 minutes to remove excess ethanol and eluted with 25 μl of ddH20.

### Example 3

Real time PCR (qPCR): qPCR was performed using 5 μL of DNA (3 ng/μL) 12.5 μL 2× LongAmp (NEB) with 1.25 μL of 10 μM 16S primers/probe and/or ITS primers/probe (i.e., MGC-ITS3F and MGC-ITS3R primers) as well as primers/probes for PsiK, PsiM, and/or PsiD. 10 μL ddH20 was used for a 25 μL total reaction volume. An initial 95° C. 5-minute denaturation step was performed followed by 40 cycles of 95° C. for 15 s and 65° C. for 90 s.

The following primers and probes were used:

16S Primers and Probes (TAC)
MGC-TAC_F:
(SEQ ID NO: 21)
TACACGACGTTGTAAAACGATCCTACGGGAGGCAGCAGT
MGC-TAC R:
(SEQ ID NO: 22)
AGGATAACAATTTCACACAGGGGACTACCAGGGTATCTAATCCTGTT
TAC-Probe:
(SEQ ID NO: 23)
/56-FAM/CGTATTACC/ZEN/GCGGCTGCTGGCAC/3IABKFQ/
Alternative: TAC-Probe:
(SEQ ID NO: 23)
/56-ROXN/CGTATTACCGCGGCTGCTGGCAC/3IAbRQSp/
ITS Primers and Probe (TYM)
MGC-ITS3F:
(SEQ ID NO: 42)
TACACGACGTTGTAAAACGACGCATCGATGAAGAACGCAGC
MGC-ITS3R:
(SEQ ID NO: 43)
AGGATAACAATTTCACACAGGATTTGAGCTCTTGCCGCTTCA
MGC-Probe:
(SEQ ID NO: 44)
/56-FAM/CGCTGAACT/ZEN/TAAGCATATCAATAAGCGG/3IABKFQ/
PsiK Primers and Probe
PsiK_Forward
(SEQ ID NO: 33)
GGAGCGAGGAAGAAAGGATAAA
PsiK_Reverse
(SEQ ID NO: 34)
GCAGTGGATGATTCCTTCAGTA
PsiK_Probe
(SEQ ID NO: 39)
/5HEX/TAGCTGCCT/ZEN/TTCACGACGCCA/3IABKFQ/
PsiM Primers and Probe
PsiM_Forward
(SEQ ID NO: 35)
CGTGGTGTTGTCTTCACTACTT
PsiM_Reverse
(SEQ ID NO: 36)
CCCATCTTGCTGAGCGTAAT
PsiM_Probe
(SEQ ID NO: 40)
/5HEX/TGGACTTGG/ZEN/GTTGCTCCTACGTG/3IABKFQ/
PsiD Primers and Probe
PsiD_Forward
(SEQ ID NO: 37)
ACCCGTCAATGGGACAATC
PsiD_Reverse
(SEQ ID NO: 38)
GAGGTGGGTCGTAATCGTTATC
PsiD_Probe
(SEQ ID NO: 41)
/5HEX/CGAGCACGA/ZEN/TTGGCGACCCTAT/3IABKFQ/

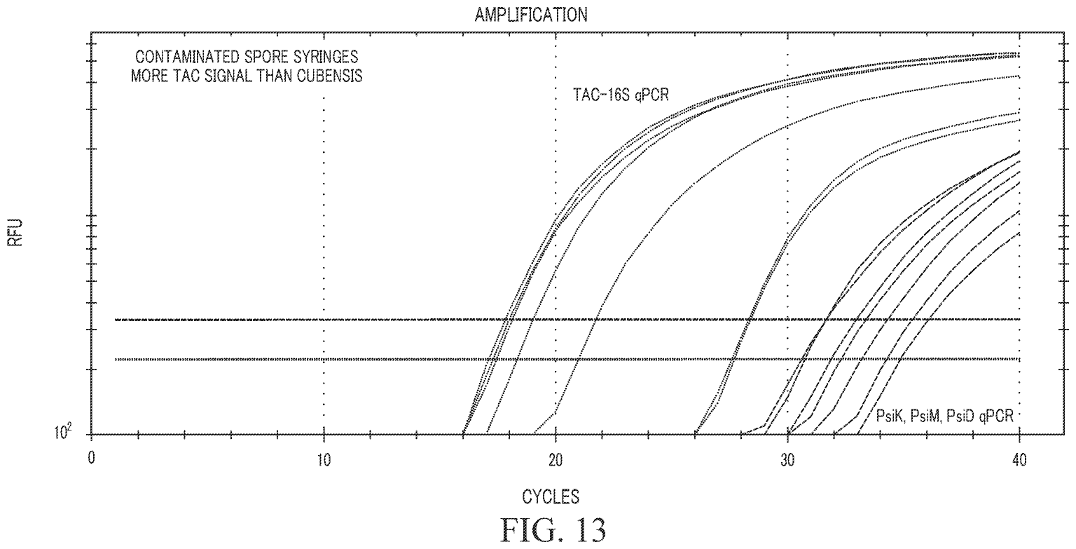

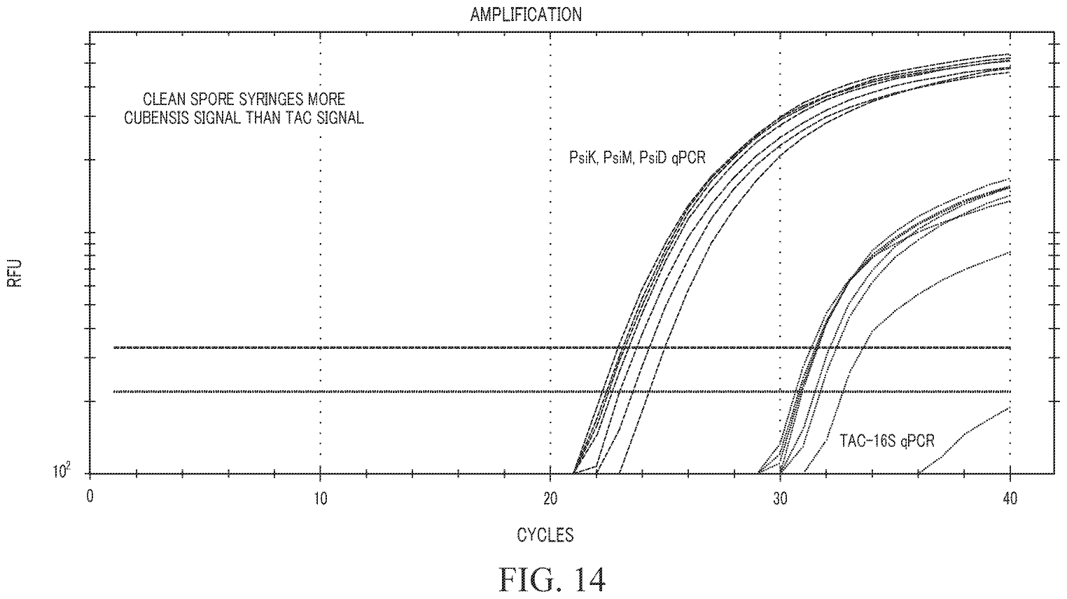

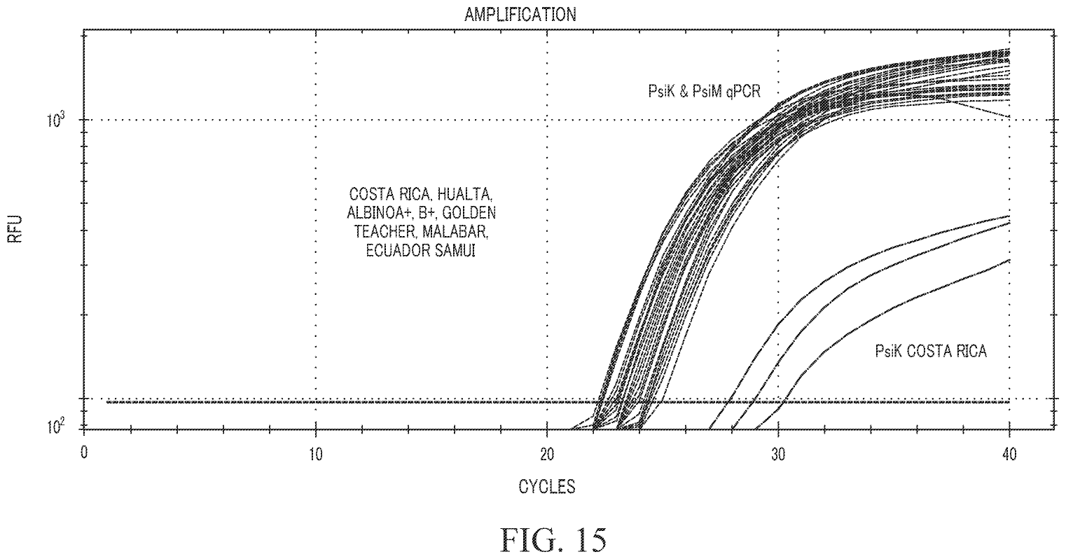

The qPCR results are shown in FIGS. 13 and 14. FIG. 13 shows qPCR results from assaying P. cubensis spores having bacterial contamination. FIG. 14 shows qPCR results from assaying P. cubensis spores that are not contaminated as compared to the qPCR results shown by FIG. 13. FIG. 15 shows qPCR results from assaying P. cubensis spores from various P. cubensis for PsiK and PsiM genes.

1. A method for discriminating Psilocybe cubensis from at least one other mushroom species subject to misidentification as Psilocybe cubensis, comprising:
obtaining a sample including nucleic acids collected from putative Psilocybe cubensis spores or tissue;
contacting the sample with primers for amplifying target nucleic acid sequences;
amplifying any of the target nucleic acid sequences when present among the nucleic acids to obtain amplicons; and
detecting the amplicons upon amplification of the target nucleic acid sequences,
wherein the primers include primers for amplifying a Psilocybe cubensis target nucleic acid sequence and a target nucleic acid sequence from the other mushroom species.

2. The method of claim 1, wherein:
the Psilocybe cubensis target nucleic acid sequence includes a Psilocybe cubensis Internal Transcribed Spacer (ITS) target nucleic acid sequence and/or any of a Psilocybe cubensis PsiK, PsiM, PsiH, or PsiD target nucleic acid sequence; and
the target nucleic acid sequence from the other mushroom species includes any of an amatoxin target nucleic acid sequence or an Internal Transcribed Spacer (ITS) target nucleic acid sequence.

3. The method of claim 1, wherein:
the Psilocybe cubensis target nucleic acid sequence includes a Psilocybe cubensis Internal Transcribed Spacer (ITS) target nucleic acid sequence and/or any of a Psilocybe cubensis PsiK, PsiM, PsiH, or PsiD target nucleic acid sequence; and
the target nucleic acid sequence from the other mushroom species includes an amatoxin target nucleic acid sequence and an Internal Transcribed Spacer (ITS) target nucleic acid sequence.

4. The method of claim 1, wherein the Psilocybe cubensis target nucleic acid sequence includes a Psilocybe cubensis Internal Transcribed Spacer (ITS) target nucleic acid sequence.

5. The method of claim 1, wherein the target nucleic acid sequence from the other mushroom species includes an amatoxin target nucleic acid sequence from an amatoxin-producing Galerina species.

6. The method of claim 1, wherein the target nucleic acid sequence from the other mushroom species includes an Internal Transcribed Spacer (ITS) target nucleic acid sequence from a Psilocybe species linked to wood lovers paralysis (WLP).

7. The method of claim 1, wherein the target nucleic acid sequence from the other mushroom species includes an Internal Transcribed Spacer (ITS) target nucleic acid sequence from any of Psilocybe azurescens, Psilocybe cyanescens, Psilocybe allenii, Psilocybe subaeruginosa, Psilocybe stunzii, Psilocybe pelliculosa, or Psilocybe ovoideocystidiata.

8. The method of claim 1, wherein the target nucleic acid sequence from the other mushroom species includes an Internal Transcribed Spacer (ITS) target nucleic acid sequence from any of Psilocybe azurescens, Psilocybe cyanescens, Psilocybe allenii, or Psilocybe subaeruginosa.

9. The method of claim 1, wherein detection of the amplicons includes quantification of the amplicons.

10. The method of claim 1, wherein the amplicons amplified from the target nucleic acid sequence from the other mushroom species are quantified during detection.

11. The method of claim 1, wherein the amplicons amplified from the Psilocybe cubensis target nucleic acid sequence are quantified during detection.

12. The method of claim 1, wherein a polymerase chain reaction (PCR) is performed to amplify the target nucleic acid sequences.

13. The method of claim 1, wherein a real-time polymerase chain reaction (qPCR) is performed to amplify the target nucleic acid sequences.

14. The method of claim 1, wherein multiplex amplification is performed to amplify the target nucleic acid sequences.

15. The method of claim 1, wherein probes are hybridized to the amplicons to detect the amplicons.

16. The method of claim 15, wherein the probes include a fluorescent label.

17-24. (canceled)

25. A kit for discriminating Psilocybe cubensis from at least one other mushroom species subject to misidentification as Psilocybe cubensis, comprising:
primers for amplifying a Psilocybe cubensis target nucleic acid sequence and a target nucleic acid sequence from the other mushroom species; and
probes for detecting amplicons of the Psilocybe cubensis target nucleic acid sequence and the target nucleic acid sequence from the other mushroom species,
wherein the probes include a fluorescent label.

26. (canceled)

27. (canceled)

28. The kit of claim 25, wherein:
the Psilocybe cubensis target nucleic acid sequence includes a Psilocybe cubensis Internal Transcribed Spacer (ITS) target nucleic acid sequence and/or any of a Psilocybe cubensis PsiK, PsiM, PsiH, or PsiD target nucleic acid sequence; and
the target nucleic acid sequence from the other mushroom species includes any of an amatoxin target nucleic acid sequence or an Internal Transcribed Spacer (ITS) target nucleic acid sequence.

29. The kit of claim 25, wherein:
the Psilocybe cubensis target nucleic acid sequence includes a Psilocybe cubensis Internal Transcribed Spacer (ITS) target nucleic acid sequence and/or any of a Psilocybe cubensis PsiK, PsiM, PsiH, or PsiD target nucleic acid sequence; and
the target nucleic acid sequence from the other mushroom species includes an amatoxin target nucleic acid sequence and an Internal Transcribed Spacer (ITS) target nucleic acid sequence.

30. The kit of claim 25, wherein the Psilocybe cubensis target nucleic acid sequence includes a Psilocybe cubensis Internal Transcribed Spacer (ITS) target nucleic acid sequence.

31-85. (canceled)
